(19) United States
(12) Patent Application Publication
(10) Pub. No.: US 2022/0257106 A1
(43) Pub. Date: Aug. 18, 2022

(54) HEAT REMOVAL INFRASTRUCTURES FOR ENDOSCOPES

(71) Applicant: 270 SURGICAL LTD., Netanya (IL)

(72) Inventors: Golan SALMAN, Atlit (IL); Amram AIZENFELD, Ramot Menashe (IL)

(21) Appl. No.: 17/614,121

(22) PCT Filed: Jun. 3, 2020
(86) PCT No.: PCT/IL2020/050613

§ 371 Date: Nov. 24, 2021

Related U.S. Application Data
(60) Provisional application No. 62/857,331, filed on Jun. 5, 2019.
(60) Provisional application No. 62/942,161, filed on Dec. 1, 2019.

(51) Int. Cl. A61B 1/12; A61B 1/00; A61B 1/06; A61B 1/05

(52) U.S. Cl. CPC A61B 1/128; A61B 1/00177; A61B 1/00096; A61B 1/05; A61B 1/06

(57) ABSTRACT

Disclosed herein is an elongated shaft for a multi-camera endoscope. The shaft includes (i) a circuit board assembly (CBA) including at least a front camera and two opposite-facing side-cameras; (ii) a heat sink structure located at a distal section of the shaft; (iii) an illumination unit(s) mounted on the heat sink structure and configured to illuminate a field-of-view of the cameras; and (iv) at least one conduit configured for heat conveyance, extending proximally along the shaft from the heat sink structure, via which the at least one conduit is thermally coupled to the illumination unit(s). The heat sink structure is essentially cylindrical and includes (i) at least two complementary parts forming a compartment therebetween that accommodates the distal section of the CBA, and (ii) holes where through lens assemblies of the cameras are fitted such as to secure the distal section of the CBA within the compartment.

### TECHNICAL FIELD

The present disclosure relates generally to heat removal infrastructures for multi-camera endoscopes.

### BACKGROUND

An endoscope is a medical device used to image an anatomical site (e.g. a body cavity, a hollow organ). Unlike some other medical imaging devices, the endoscope is inserted into the anatomical site (e.g. through small incisions made on the skin of the patient). An endoscope can be employed not only to inspect an anatomical site and e.g. organs therein (and diagnose a medical condition in the anatomical site) but also as a visual aid in surgical procedures. Medical procedures involving endoscopy include laparoscopy, arthroscopy, cystoscopy, ureteroscopy, and hysterectomy.

### SUMMARY

Aspects of the disclosure, according to some embodiments thereof, relate to heat removal infrastructures for multi-camera endoscopes. More specifically, but not exclusively, aspects of the disclosure, according to some embodiments thereof, relate to heat removal infrastructures for rigid and semi-rigid multi-camera endoscopes. The heat removal infrastructure includes: (i) a heat sink structure at a distal section of the shaft of the endoscope, the shaft having mounted thereon illumination units configured to illuminate a field-of-view (FOV) of the cameras, and (ii) a heat conduit which is thermally coupled to the heat sink structure and extends along the shaft. The heat removal infrastructure is configured to conduct away from the distal section of the shaft heat generated by electrical components therein, such as the cameras, electronic circuitry, and, in particular, the illumination units, thereby helping to prevent (i) heat-resultant damage (e.g. burns) to a subject undergoing an endoscopy procedure, and (ii) heat-induced noise disturbances to the images/videos obtained by the cameras. Advantageously, the heat sink structure is further configured to accommodate and secure the cameras and related electronics within the distal section of the shaft. According to some embodiments, the heat sink structure is made of two metallic complementary parts which are welded together and, as such, is both robust and durable. Further, according to some embodiments, side-illumination modules (e.g. light-emitting diodes on the side-illumination units) are not positioned back-to-back, so that the shaft distal section may be kept narrow.

Thus, according to an aspect of some embodiments, there is provided an elongated shaft for a multi-camera rigid endoscope. The shaft includes:
A circuit board assembly (CBA) including, on a distal section thereof, at least a front camera and two opposite-facing side-cameras configured to jointly provide a field-of-view (FOV) of at least about 270 degrees;
A heat sink structure located at a distal section of the shaft.
One or more illumination units mounted on the heat sink structure and configured to illuminate the FOV.
At least one conduit configured for heat conveyance. The at least one conduit is thermally coupled to the heat sink structure, and thereby to the one or more illumination units. The at least one conduit extends proximally from the heat sink structure along the shaft or a part of the shaft,

The heat sink structure may be essentially cylindrical and includes at least two complementary parts forming a compartment therebetween that accommodates the distal section of the CBA. The heat sink structure includes holes where through lens assemblies of the cameras are fitted, respectively, such as to secure the distal section of the CBA within the compartment.

According to some embodiments, at least one of the holes is formed by corresponding grooves on the complementary parts.

According to some embodiments, the complementary parts of the heat sink structure are metallic and welded onto one another, glued onto one another (e.g. using epoxy glue), and/or secured onto one another using screws.

According to some embodiments, the two opposite-facing side-cameras include a first a side-camera and a second side-camera. The first side-camera and the second side-camera are positioned on the CBA such that a distance of the first side-camera to a distal end of the shaft is greater than a distance of the second side-camera to the distal end. (That is, the first side-camera and the second side-camera are not positioned back-to-back.)

According to some embodiments, the front camera is offset relative to a longitudinal axis of the shaft, which centrally extends along the shaft.

According to some embodiments, the one or more illumination units include one or more light emitting diodes (LEDs).

According to some embodiments, the one or more illumination units include a front illumination unit and two side-illumination units. The front illumination unit is mounted on a distal end of the heat sink structure. The heat sink structure includes on opposite side-surfaces thereof two niches in which the two side illumination units are mounted, respectively.

According to some embodiments, the front illumination unit is attached to the distal end of the shaft and wherein the two side illumination units are attached to the side-surfaces of the of the heat sink structure in the two niches, respectively.

According to some embodiments, the front illumination unit is glued, screwed, and/or brazed with tin, to the distal end of the shaft. The two side illumination units are glued, screwed, and/or brazed with tin to the side-surfaces of the of the heat sink structure in the two niches, respectively.

According to some embodiments, the shaft further includes a rigid cover mounted on the heat sink structure and an elongated tube wherethrough the at least one conduit extends.

The cover includes windows positioned over the lens assemblies and the illumination units. The elongated tube is connected at a distal end thereof to a proximal end of the cover, and wherein the connection is fluid tight such as to fluidly seal the distal section of the shaft.

According to some embodiments, the cover is made of stainless steel.

According to some embodiments, the cover and the tube are connected by laser welding.

According to some embodiments, the at least two complementary parts of the heat sink structure include a first part and a second part, each being shaped essentially as a half cylinder.

According to some embodiments, the first part includes an opening along the length thereof, and wherein the second part includes a depression on an inner surface thereof, the depression and the opening forming the compartment of the heat sink structure.

According to some embodiments, the CBA includes a neck member connecting the distal section of the CBA to a proximal section of the CBA. The first part includes, on an outer surface of a proximal section of the first part, a recess (e.g. an elongated channel) wherethrough the neck member extends.

According to some embodiments, the at least one conduit consists of a single heat conduit. The heat sink structure includes a socket extending in the distal direction from the proximal end of the heat sink structure. A distal portion of the conduit is fitted into the socket, such as to thermally couple the heat conduit to the heat sink structure.

According to some embodiments, the socket is formed by elongated grooves on the first part and/or the second part of the heat sink structure.

According to some embodiments, the heat conduit is made of, or includes, a material characterized by high thermal conductance.

According to some embodiments, the heat conduit is metallic.

According to some embodiments, the heat conduit is non-hollow.

According to some embodiments, the at least one conduit includes two conduits. The two conduits are hollow and extend in parallel to one another. The heat sink structure includes a first channel and a second channel. The first channel and the second channel each extend in the proximal direction from a proximal end of the heat sink structure. The first channel and the second channel are fluidly coupled to one another via respective distal ends thereof. The two conduits are further fluidly connected to first channel and the second channel, respectively. The shaft is thereby configured for fluid conveyance through the distal section of the shaft.

According to some embodiments, the heat sink structure further includes a front channel, which fluidly couples the two channels. The front channel may be positioned within the heat sink structure proximally to the distal end of the heat sink structure.

According to some embodiments, the channels are included in the second part of the heat sink structure.

According to some embodiments, wherein the first channel and the second channel, and, optionally, the front channel, are embedded in the heat sink structure.

According to an aspect of some embodiments, there is provided an endoscope including a shaft, as described above, and a handle on which the shaft is mounted.

According to some embodiments wherein the shaft includes the two conduits and the first channel and the second channel, the two conduits include a first conduit and a second conduit, and the handle includes a fluid reservoir and a pump. The fluid reservoir is fluidly coupled to the two conduits. The pump is configured to circulate fluid from the fluid reservoir by pumping fluid into the first conduit and drawing fluid from the second conduit.

According to some embodiments wherein the shaft includes the two conduits and the first channel and the second channel, the handle further includes a fluid-intake port and a fluid-expulsion port. A first conduit, from the two conduits, is fluidly coupled to the fluid-intake port and a second conduit, from the two conduits, is fluidly coupled to the fluid-expulsion port.

According to some embodiments, the handle further includes a pump configured for drawing fluid into the fluid-intake port and circulating the fluid via the two conduits, and the channels.

According to some embodiments, the handle or the second conduit comprises a thermometer configured to measure a temperature of fluid which has exited the distal section of the shaft.

According to an aspect of some embodiments, there is provided an elongated semi-rigid shaft for a multi-camera endoscope. The semi-rigid shaft includes an at least partially hollow rigid elongated member, a distal tip portion, and a maneuvering portion mounted between and mechanically coupling the rigid elongated member to the distal tip portion. The distal tip portion includes:
A circuit board assembly (CBA) including, on a distal section thereof, at least a front camera and two opposite-facing side-cameras configured to jointly provide a field-of-view (FOV) of at least about 270 degrees.
A heat sink structure.
One or more illumination units mounted on the heat sink structure and configured to illuminate the FOV.

The semi-rigid shaft further includes at least one semi-rigid conduit configured for heat conveyance. The at least one semi-rigid conduit is thermally coupled to the heat sink structure, and thereby to the one or more illumination units. The at least one semi-rigid conduit extends proximally from the heat sink structure along the semi-rigid shaft or a part of the semi-rigid shaft. The at least one semi-rigid conduit is configured to be bent and/or twisted in accordance with the maneuvering portion. The heat sink structure may be essentially cylindrical and includes at least two complementary parts forming a compartment therebetween that accommodates the distal section of the CBA. The heat sink structure includes holes wherethrough lens assemblies of the cameras are fitted, respectively, such as to secure the distal section of the CBA within the compartment.

According to some embodiments, the two opposite-facing side-cameras include a first a side-camera and a second side-camera. The first side-camera and the second side-camera are positioned on the CBA such that a distance of the first side-camera to a distal end of the semi-rigid shaft is greater than a distance of the second side-camera to the distal end.

According to some embodiments, the at least one semi-rigid conduit includes two semi-rigid conduits, which are hollow and extend in parallel to one another. The heat sink structure includes a first channel and a second channel. The first channel and the second channel each extend in the distal direction from a proximal end of the heat sink structure. The first channel and the second channel are fluidly coupled to one another via respective distal ends thereof. The two semi-rigid conduits are fluidly connected to the first channel and the second channel, respectively. The semi-rigid shaft being thereby configured for fluid conveyance through the distal section of the semi-rigid shaft.

According to some embodiments, a distal section of the heat sink structure includes a front channel fluidly coupling the first channel and the second channel.

According to an aspect of some embodiments, there is provided an endoscope including a semi-rigid shaft, as described above, and a handle on which the semi-rigid shaft is mounted.

According to some embodiments wherein the semi-rigid shaft includes the two semi-rigid conduits and the first channel and the second channel, the handle includes a fluid reservoir and a pump. The fluid reservoir is fluidly coupled to the two semi-rigid conduits. The pump is configured to circulate fluid to and from the fluid reservoir via the two semi-rigid conduits, respectively.

According to some embodiments wherein the semi-rigid shaft includes the two semi-rigid conduits and the first channel and the second channel, the handle includes a fluid-intake port and a fluid-expulsion port. A first conduit, from the two semi-rigid conduits, is fluidly coupled to the fluid-intake port, and a second conduit, from the two-semi-rigid conduits, is fluidly coupled to the fluid-expulsion port.

According to some embodiments, the handle further includes a pump configured for drawing fluid into the fluid-intake port and for circulating the fluid via the two semi-rigid conduits and the channels.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1 presents a schematic, perspective view of a rigid endoscope including an elongated shaft mounted on a handle, according to some embodiments;

FIG. 2 schematically depicts a medical imaging system including the rigid endoscope of FIG. 1, according to some embodiments;

FIGS. 3A and 3B present schematic, perspective views of an optical unit of the rigid endoscope of FIG. 1, the optical unit including cameras mounted on a circuit board assembly, according to some embodiments;

FIGS. 4A and 4B schematically depict a heat sink structure of the rigid endoscope of FIG. 1 with the optical unit of FIG. 3A inserted into the heat sink structure, according to some embodiments;

FIGS. 5A and 5B are schematic, perspective views of a lower part of the heat sink structure of FIG. 4A, according to some embodiments;

FIG. 6 is a schematic, perspective view of an upper part of the heat sink structure of FIG. 4A, according to some embodiments;

FIGS. 7A and 7B schematically depict the heat sink structure of FIG. 4A with illumination units mounted thereon (and with the optical unit of FIG. 3A inserted thereinto), according to some embodiments;

FIG. 8 schematically depicts the heat sink structure of FIG. 7A with a rigid cover mounted thereon, according to some embodiments;

FIG. 9 schematically depict the heat sink structure of FIG. 8 (i.e. the heat sink structure of FIG. 7A with the rigid cover mounted thereon) with window components installed on a distal tip of the heat sink structure and side-openings on the rigid cover, according to some embodiments;

FIGS. 10A and 10B depict the heat sink structure of FIG. 9 (including the rigid cover and the window components) with a heat conduit inserted thereinto, according to some embodiments;

FIGS. 11A and 11B present schematic, perspective views of the distal section and a central section of the rigid shaft of FIG. 1, according to some embodiments;

FIG. 12 presents a schematic, perspective view of a rigid endoscope including an elongated shaft mounted on a handle, according to some embodiments;

FIG. 13 presents a schematic, exploded view of a heat sink structure of the rigid endoscope of FIG. 12 with an optical unit of the endoscope inserted into the heat sink structure, according to some embodiments;

FIG. 14 presents a schematic, perspective, semi-transparent view of an upper part of the heat sink structure of FIG. 13, according to some embodiments;

FIG. 15 presents a schematic, perspective view of the upper part of the heat sink structure of FIG. 14 with two heat conduits inserted thereinto, according to some embodiments;

FIGS. 16A and 16B present perspective, cross-sectional views of the upper part of the heat sink structure of FIG. 14 (and the two heat conduits of FIG. 15), according to some embodiments;

FIG. 17 presents a schematic, perspective view of a lower part of the heat sink structure of FIG. 13, according to some embodiments;

FIG. 18 presents a schematic, perspective view of the heat sink structure of FIG. 13 (including a rigid cover and window components and with the two heat conduits of FIG. 15 inserted thereinto), according to some embodiments; and

FIG. 19 schematically depicts a medical imaging system including the rigid endoscope of FIG. 12 and a fluid circulation system, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

Referring to the figures, in flowcharts, optional steps may appear within boxes delineated by a dashed line. Similarly, in block diagrams, optional elements/components may be delineated by a dashed line.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 99% and 101% of the given value. For example, the statement “the length of the element is equal to about 1 millimeter” is equivalent to the statement “the length of the element is between 0.99 millimeters and 1.01 millimeters”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “essentially”, “substantially” and “about” may be interchangeable.

For ease of description, in some of the figures a three-dimensional cartesian coordinate system (with orthogonal axes x, y, and z) is introduced. It is noted that the orientation of the coordinate system relative to a depicted object may vary from one figure to another.

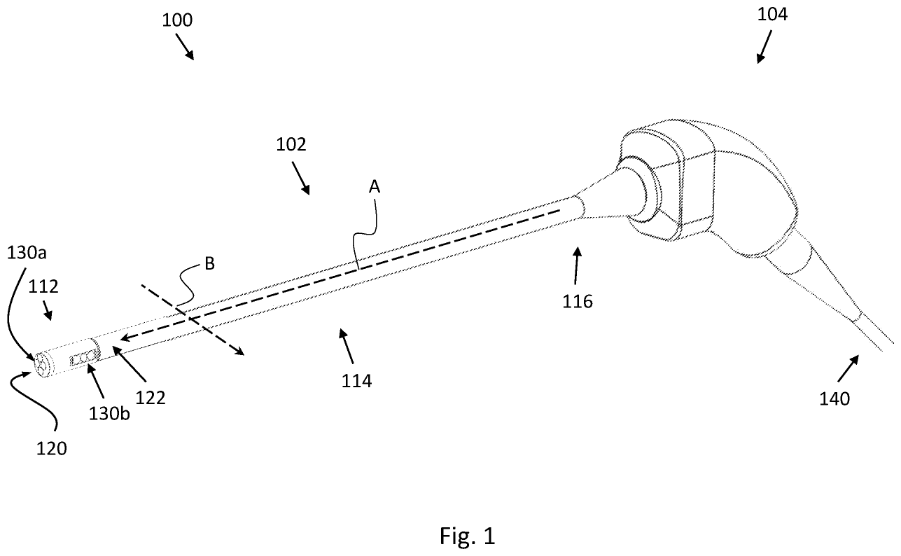

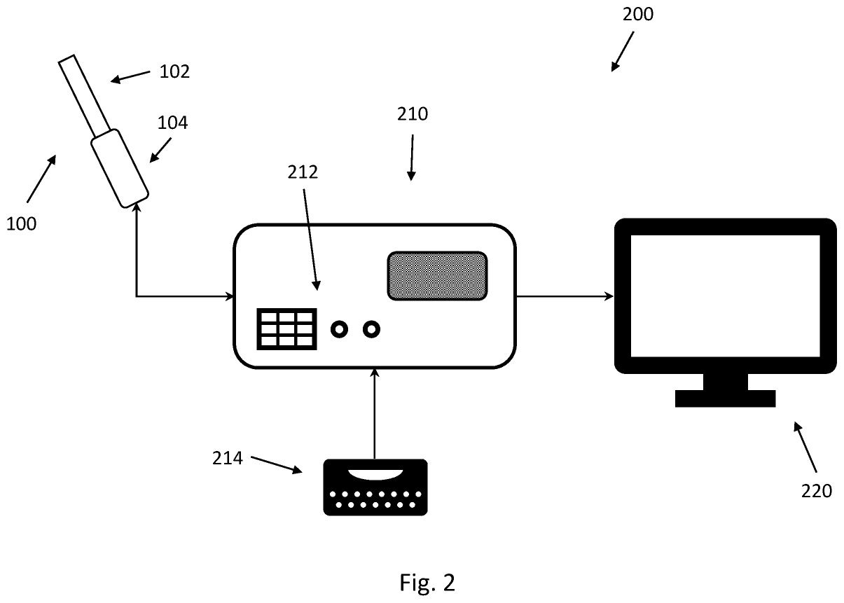

According to an aspect of some embodiments, schematically depicted in FIGS. 1 and 3A-11B, there is provided an endoscope. FIG. 1 schematically depicts such an endoscope, an endoscope 100, according to some embodiments. FIG. 2 schematically depicts an image monitoring system including endoscope 100.

It is to be understood that endoscope 100 may be a rigid endoscope, as depicted in FIG. 1, or a semi-rigid endoscope, as elaborated on below. Endoscope 100 includes an elongated shaft 102, configured to be inserted into an anatomical site (e.g. an anatomical cavity), and a handle 104, configured to be held by a user (e.g. a surgeon) of endoscope 100 and to facilitate guidance and manipulation of shaft 102 (particularly a distal section thereof) within the anatomical site. Shaft 102 is mounted on handle 104. According to some embodiments, shaft 102 is integrated in endoscope 100 as an extension of handle 104. According to some embodiments, shaft 102 is configured to be detachably mountable on handle 104.

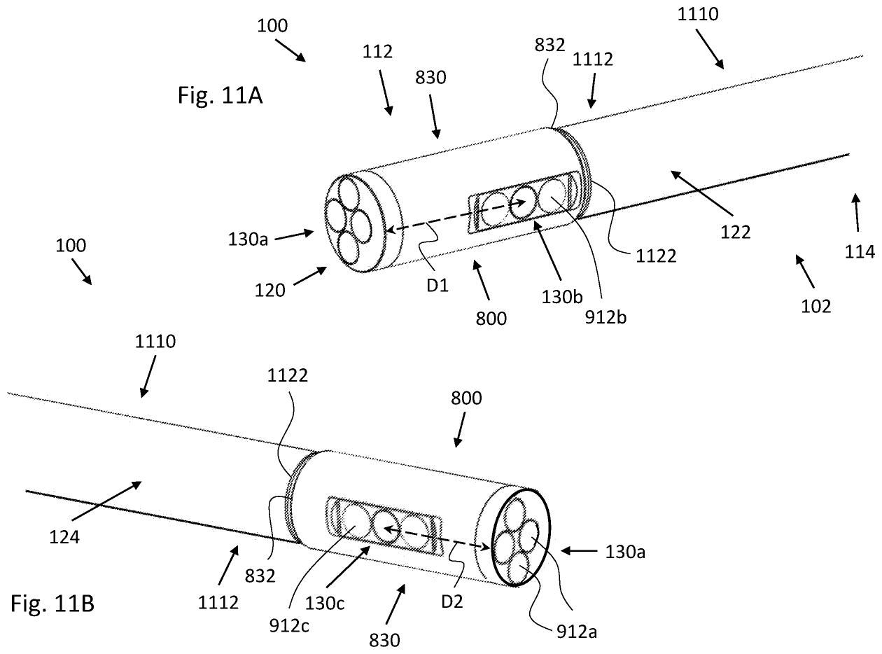

Shaft 102 includes a shaft distal section 112, a shaft central section 114, and a shaft proximal section 116 (i.e. a distal section, a central section, and a proximal section, respectively, of shaft 102), a shaft distal tip 120 (whereat shaft distal section 112 terminates in the distal direction), a shaft first side-surface 122, and a shaft second side-surface 124 (shown in FIG. 11B). A longitudinal axis A, pointing in the distal direction, and along which shaft 102 extends, is also indicated. Further indicated is transverse axis B, which is perpendicular to the longitudinal axis A.

According to some embodiments, shaft 102 may have a diameter measuring between about 2 millimeters and about 15 millimeters. Shaft distal section 112 may measure between about 1 millimeter and about 25 millimeters in length. According to some embodiments, shaft distal section 112 may have a narrower diameter than the rest of shaft 102, as described in PCT application publication No. WO2019035118 to A. Levy et al., which is incorporated herein by reference in its entirety.

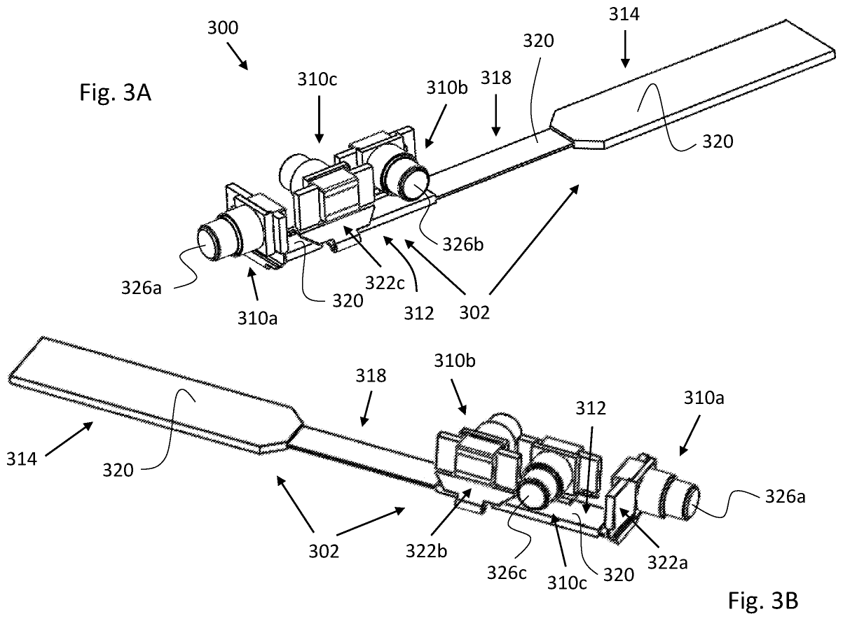

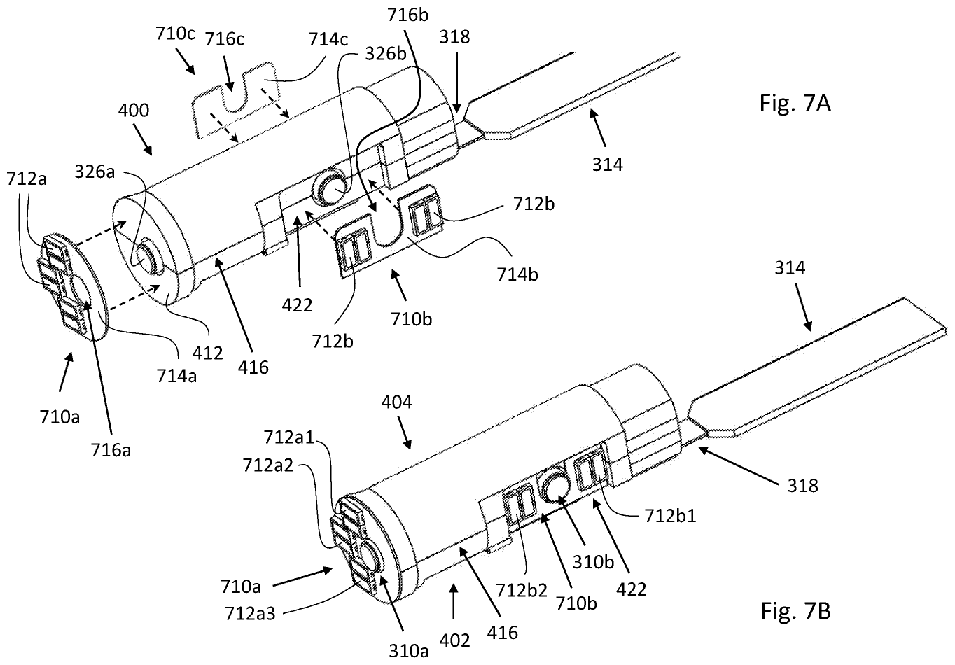

Shaft distal section 112 houses at least three cameras 310 (shown in FIGS. 3A and 3B), and one or more illumination units 710 (for example, three, as shown in FIGS. 7A and 7B) associated with cameras 310. Cameras 310 and illumination units 710 are positioned behind window components 130, which protect cameras 310 and illumination units 710 from e.g. body fluids and debris during an endoscopy procedure. Further, shaft distal section 112 may be fluidly sealed such as to prevent air/gas from entering therein during an endoscopy procedure and when undergoing cleaning. (Air penetration may lead to the formation of a condensate on the lenses of cameras 310 and the inner surface of the windows of window components 130, and thereby blur video and images obtained by cameras 310. Further, moisture may lead to corrosion of electrical components within shaft distal section 112, which may result in malfunctioning of cameras 130 and illumination units 710. During manufacture, inert gas, such as nitrogen, may be pumped into shaft distal section 112, prior to the sealing thereof.) Illumination units 710, which may include light sources, such as light emitting diodes (LEDs), are configured to illuminate the field-of-view of cameras 310, as elaborated on below. Illumination units 710 may generate significant amounts of heat during operation, so that efficient removal of heat may be required in order to protect a subject, undergoing endoscopy, from heat-resultant wounds.

According to some embodiments, not depicted in the figures, illumination units 710 include the distal tips of respective optical fibers. According to some such embodiments, handle 104 may include one or more light sources connected to one or more optical fibers extending through handle 104 and shaft 102. The optical fibers are configured to guide the light produced by the light sources from handle 104 to shaft distal section 112, wherefrom the guided light may be shone such as to illuminate the field-of-view of cameras 310. According to some embodiments, the light sources may be external to handle 104, being positioned, for example, in a main control unit such as the main control unit depicted in FIG. 2.

Handle 104 may include a user control interface (not shown) configured to allow a user to control endoscope 100 functions. In particular, the user control interface is functionally associated with cameras 310 and illumination units 710. According to some embodiments, the user control interface may allow, for example, to control zoom, focus, record/stop recording, and/or freeze frame functions of cameras 310 and/or to adjust the intensity of light provided by illumination units 710 collectively and/or individually. The user control interface may include one or more buttons, knobs, switches, a touch panel, and/or the like.

According to some embodiments, endoscope 100 may be (i) directly maneuvered by a user through the manipulation of handle 104, as well as (ii) indirectly maneuvered, via robotics, e.g. using a robotic arm or other suitable gripping means configured to allow manipulation of handle 104.

FIG. 2 schematically depicts a medical imaging system 200, according to some embodiments. Medical imaging system 200 includes endoscope 100, a main control unit 210, and a monitor 220. Endoscope 100 and monitor 220 are each functionally associated with main control unit 210. Main control unit 210 includes electronic circuitry (e.g. one or more processors and memory components) configured to process (digital data) from cameras 310 such as to display the captured images and video(s) on monitor 220. In particular, the processing circuitry may be configured to process the digital data received from each of cameras 310 such as to produce therefrom a combined video file/stream providing a continuous and consistent (seamless) panoramic view of an anatomical site wherein endoscope 100 is inserted.

Main control unit 210 may include a user control interface 212 (e.g. buttons and/or knobs, a touch panel, a touch screen) configured to allow a user to operate main control unit 210, or may allow control thereof using one or more input devices 214, e.g. an external user control interface connectable thereto, such as a keyboard, a mouse, a portable computer, and/or a mobile computational device, such as a smartphone or a tablet. According to some embodiments, input devices 214 may include a voice controller. According to some embodiments, main control unit 210 may further be configured to partially or even fully operate cameras 310 and illumination units 710. Some operational aspects may be operated automatically. For example, according to some embodiments, the supply of power to endoscope 100 components, such as cameras 310 and illumination units 710 may be operated automatically, while other operational aspects or functions may be operated using the user control interface included in main control unit 210, and/or the external user control interface connectable thereto.

According to some embodiments, endoscope 100 is functionally associated with main control unit 210 via a utility cable 140 (shown in FIG. 1) configured to be connected to handle 104. Cable 140 may include at least one data cable for receiving video signals from cameras 310, as well as at least one power cable for providing electrical power to cameras 310 and illumination units 710. Additionally/alternatively, according to some embodiments, endoscope 100 includes a wireless communication unit (e.g. a Bluetooth antenna), which may be configured to communicatively associate endoscope 100 with main control unit 210. According to some embodiments, endoscope 100 is configured to be powered by a replaceable and/or rechargeable battery included therein, i.e. inside handle 104.

FIGS. 3A and 3B schematically depict an optical unit 300 including a circuit board assembly 302 (CBA; e.g. a printed circuit board and electronic components, such as processors, amplifiers, and discrete components) and cameras 310, according to some embodiments. Cameras 310 are positioned on CBA 302. CBA 302 may be or include a printed circuit board (PCB). According to some embodiments, CBA 302 includes a board distal section 312, on which cameras 310 are positioned, and a board proximal section 314. According to some embodiments, board distal section 312 and board proximal section 314 are joined to one another by a board neck member 318, which may have smaller lateral dimensions than both board distal section 312 and board proximal section 314. A board top surface 320—including the top surfaces of board distal section 312, board proximal section 314, and board neck member 318—is also indicated.

According to some embodiments, CBA 302 is foldable. For example, and as depicted in FIGS. 3A and 3B, board distal section 312 may include foldable arms 322: a front arm 322a, a first side-foldable arm 322b, and a second side-foldable arm 322c. Cameras 310 are mounted on foldable arms 322.

According to some embodiments, and as depicted in FIG. 1, cameras 310 are positioned, or substantially positioned, on the plane defined by longitudinal axis A and transverse axis B.

Cameras 310 include a front camera 310a, a first side-camera 310b, and a second side-camera 310c. Each of cameras 310 may include a sensor, such as a charge coupled device (CCD) image sensor or a complementary metal oxide semiconductor (CMOS) image sensor, and a lens assembly (e.g. including an extreme wide-angle camera lens). As elaborated on below, optical unit 300 is configured to be installed within shaft distal section 112 such that: (i) a front lens assembly 326a of front camera 310a is positioned at, or substantially at, shaft distal tip 120 facing forward in the direction of longitudinal axis A; (ii) a first side-lens assembly 326b of first side-camera 310b is positioned at, or substantially at, shaft first side-surface 122 and faces sideways along the direction, or substantially along the direction, defined by the transverse axis B; and (iii) a second side-lens assembly 326c of second side-camera 310c is positioned at, or substantially at, shaft second side-surface 124 and faces side-ways in the opposite sense, or substantially in the opposite sense, to the direction defined by the arrow of transverse axis B (i.e. in direction of an arrow pointing at 180 degrees to the arrow on transverse axis B). According to some embodiments, first side-camera 310b and second side-camera 310c are not oriented in (exactly) opposite directions. As a non-limiting example, first side-camera 310b may be oriented along a first axis subtending a first angle from the longitudinal axis A, which is slightly different than 90 degrees (as a non-limiting example, between 87 degrees and 89 degrees), and, similarly, second side-camera 310c may be oriented along a second axis subtending a second angle from the longitudinal axis A, which is slightly different than 90 degrees. According to some embodiments, both the first angle and the second angle may be smaller than 90 degrees, so that the angle defined by the orientations of first side-camera 310b and second side-camera 310c is smaller than 180 degrees, and each of side-cameras 310b and 310c is slightly tilted towards the distal direction. According to some other embodiments, the first angle may be smaller than 90 degrees while the second angle may be greater than 90 degrees. According to some such embodiments, first side-camera 310b and second side-camera 310c point along opposite directions (so as to be slightly tilted relative to the transverse axis B).

According to some embodiments, cameras 310 are configured to provide, in combination, a continuous field-of-view (FOV) of at least about 270 degrees. More specifically, the horizontal FOV provided by cameras 310 may be at least about 270 degrees, wherein the horizontal plane is defined (i.e. spanned) by the axes A and B. The positioning of cameras 310 within shaft distal section 112 may be selected such as to minimize the space occupied by cameras 310 and reduce the diameter of shaft distal section 112 and the whole of shaft 102, while affording a continuous FOV at least about 270 degrees.

It is noted that in the figures first side-camera 310b and second side-camera 310c are not positioned back-to-back on board distal section 312. That is, when board distal section 312 and cameras 310 are installed within shaft distal section 112, first side-camera 310b is positioned at a distance D1 (indicated in FIG. 11A) from shaft distal tip 120, and second side-camera 310c is positioned at a distance D2 (indicated in FIG. 11B) from shaft distal tip 120, with D1>D2. In particular, first side-camera 310b and second side-camera 310c may be positioned adjacently to one another along longitudinal axis A, thereby saving space and helping to restrict the lateral dimensions (i.e. the diameter) of shaft distal section 112 to below about 11 millimeters. According to some embodiments, the distance D1 may be between about 10 millimeters and about 27 millimeters, and the distance D2 may be between about 10 millimeters and about 17 millimeters. According to some embodiments, the distance D1 is between about 4 millimeters and about 15 millimeters, and the distance D2 is between about 4 millimeters and about 15 millimeters, but at the same time D2 may be smaller than D1.

According to some embodiments, not depicted in the figures, first side-camera 310b and second side-camera 310c are positioned back-to-back on the plane defined by the longitudinal axis A and the transverse axis B, and on opposite sides of the longitudinal axis A.

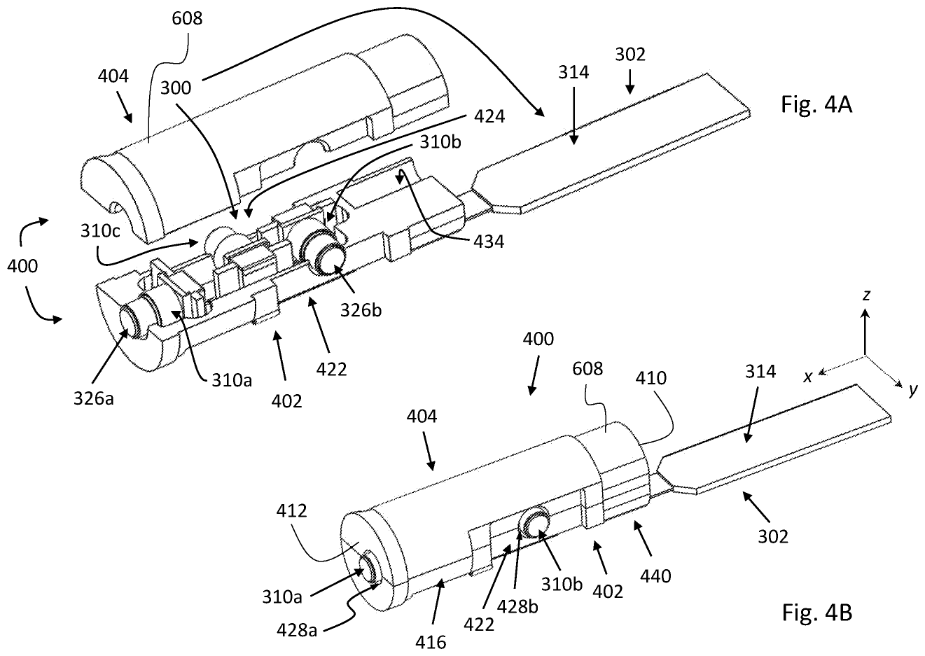

FIGS. 4A and 4B schematically depict a heat sink structure 400, according to some embodiments. Heat sink structure 400 is configured to accommodate therein board distal section 312 (and cameras 310) and to have mounted thereon illumination units 710. Heat sink structure 400 is further configured to absorb heat generated primarily by illumination units 710, and to expel the heat, as explained below.

According to some embodiments, heat sink structure 400 includes a sink lower part 402 and a sink upper part 404 (i.e. a lower part and an upper part of heat sink structure 400), which are joined onto one another, e.g. by welding and/or gluing (typically with epoxy) and/or using screws. FIG. 4A is a schematic, exploded view of heat sink structure 400 with board distal section 312 and cameras 310 accommodated therein. More specifically, FIG. 4A depicts sink lower part 402 and sink upper part 404 “separately” from one another, with board distal section 312 and cameras 310 installed on/in/within sink lower part 402, as explained below. FIG. 4B is a schematic, perspective view of heat sink structure 400 (i.e. with sink lower part 402 and sink upper part 404 joined onto one another) with optical unit 300 inserted therein. Board distal section 312 and cameras 310 are installed within heat sink structure 400 (and are therefore hidden from view in FIG. 4B), as elaborated on below, and board proximal section 314 extends in the proximal direction from a sink proximal end 410 (i.e. a proximal end of heat sink structure 400).

It will be understood that other options for joining sink lower part 402 and sink upper part 404 are possible. For example, according to some embodiments, sink lower part 402 and sink upper part 404 are affixed to one another by fasteners such as nuts and bolts and/or the like.

As used herein, according to some embodiments, the terms “upper” and “lower” are used to facilitate distinguishing between elements according to the relative positions thereof in the figures.

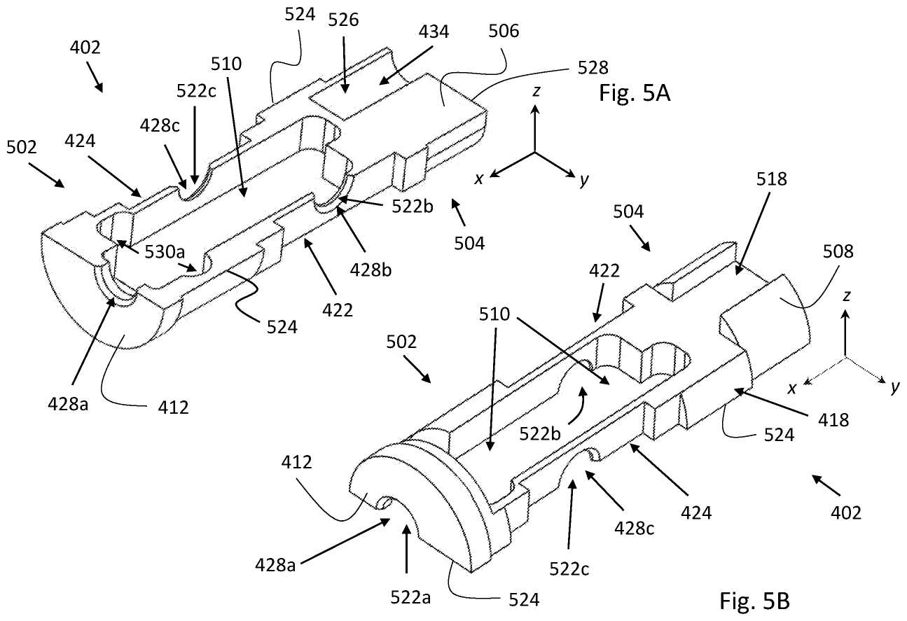

Heat sink structure 400 (distally) extends from sink proximal end 410 to a sink distal end 412 and includes a first side-surface 416 and a second side-surface 418 (partially indicated in FIG. 5B). According to some embodiments, heat sink structure 400 includes an elongated opening 510 (hidden from view in 4B; shown in full in FIGS. 5A and 5B) extending along a length (i.e. part of the total length) of heat sink structure 400 on sink lower part 402. Apart from elongated opening 510, and from niches and indentations specified below, heat sink structure 400 may generally (essentially) be cylindrically shaped. According to some embodiments, elongated opening 510 may be substantially rectangular. The function of elongated opening 510 is described below. Heat sink structure 400 includes a first side-niche 422 and a second side-niche 424 (partially indicated in FIGS. 5A and 5B), each forming a shallow indentation on first side-surface 416 and second side-surface 418, respectively. Each of first side-niche 422 and second side-niche 424 is configured (i.e. shaped and dimensioned) to accommodate one of illumination units 710, respectively, as described below. According to some embodiments, first side-niche 422 and second side-niche 424 may be rectangular or essentially rectangular.

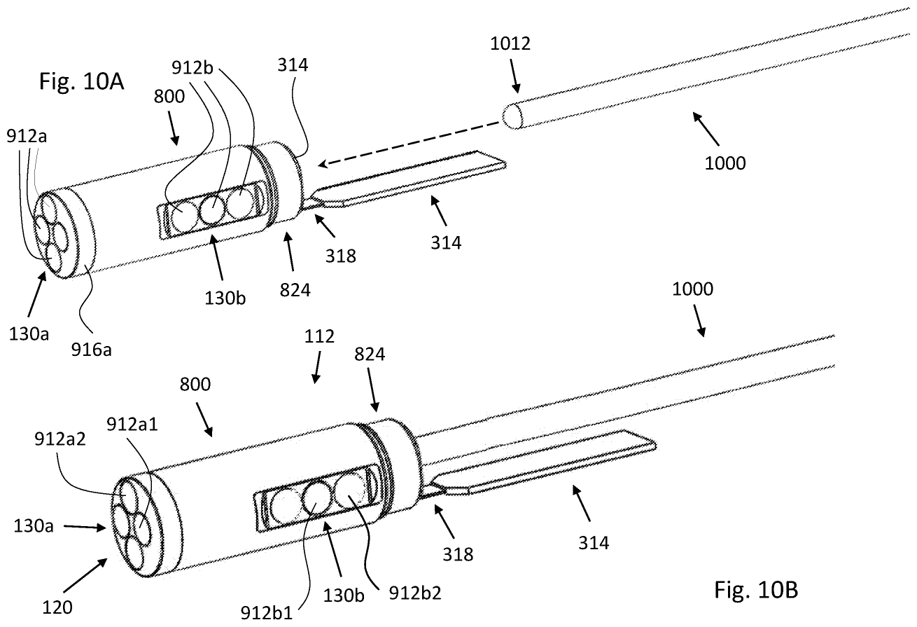

Sink distal end 412 includes a front hole 428a configured to accommodate front lens assembly 326a of front camera 310a, e.g. as shown in the figures. First side-niche 422 includes a first side-hole 428b configured to accommodate first side-lens assembly 326b of first side-camera 310b, e.g. as shown in the figures. Second side-niche 424 includes a second side-hole 428c configured to accommodate second-side lens assembly 326c of second side-camera 310c, e.g. as shown in the figures. Heat sink structure 400 further includes a socket 434 (a lower portion of which is indicated in FIG. 4A) distally extending into heat sink structure 400 from sink proximal end 410. Socket 434 is configured to receive therein a heat conduit for expelling heat from heat sink structure 400, as explained below in the description of FIGS. 10A and 10B.

Heat sink structure 400 may be made of a material which is good thermal conductor, such as, but not limited to, copper, gold, silver, aluminum, and the like, and/or an alloy including combinations thereof. According to some embodiments, heat sink structure 400 includes one or more highly thermally conductive routes from sink distal end 412, first side-niche 422, and second side-niche 424 to socket 434 (i.e. continuous pathways on and along heat sink structure 400 along which the heat conductivity is high).

According to some embodiments, a sink proximal section 440 (a proximal section of heat sink structure 400, which includes sink proximal end 410) is narrower than the rest of heat sink structure 400 for reasons which are explained below.

Making reference again also to FIGS. 3A and 3B, according to some embodiments, when board distal section 312 and cameras 310 are installed within shaft distal section 112, front camera 310a may be offset relative to the longitudinal axis A. According to some embodiments, front camera 310a may be offset by between about 0.05 millimeters and about 0.5 millimeters, between about 0.5 millimeters and about 2.5 millimeters, or between about 1 millimeter and about 5 millimeters relative to the longitudinal axis A.

Each possibility corresponds to separate embodiments. According to some embodiments, front camera 310a may be off-set relative to the longitudinal axis A towards first side-surface 416 such as to provide a continuous and seamless combined panoramic view from cameras 310a, 310b, and 310c. More specifically, the degree of offsetting of first camera 310a may be selected such as to provide seamless stitchings of the FOV of front camera 310a to the FOV of first side-camera 310b, and the FOV of front camera 310a to the FOV of second-side camera 310c, as described in PCT application publication No. WO2019030747 to A. Levy et al., which is incorporated herein by reference in its entirety.

FIGS. 5A and 5B schematically depict sink lower part 402, according to some embodiments. Sink lower part 402 includes a distal section 502, a proximal section 504, a lower part inner surface 506, which as depicted in the figures may be flat or substantially flat, and a lower part outer surface 508, which as depicted in the figures may be curved. is positioned below lower part inner surface 506. Proximal section 504 constitutes the lower part of sink proximal section 440, and as such may be narrower than distal section 502 (which is the lower part of the distal section of sink lower part 402). According to some embodiments, sink lower part 402 may generally be shaped as a half-cylinder with elongated opening 510 longitudinally extending along distal section 502 and, optionally, along a distal part of proximal section 504. Lower part outer surface 508 is longitudinally indented along proximal section 504, such as to form a recess 518 (e.g. an elongated channel) configured to accommodate board neck member 318, and to restrict both lateral (defined by they-axis in FIGS. 5A and 5B) and vertical (defined by the z-axis in FIGS. 5A and 5B) motion thereof, thereby helping to secure board distal section 312 and cameras 310 within heat sink structure 400.

Sink lower part 402 includes three grooves 522 positioned on a perimeter 524 of lower part inner surface 506: a front groove 522a, a first side-groove 522b, and a second side-groove 522c. Each of grooves 522, together with a corresponding groove on sink upper part 404, define a hole from holes 428, respectively (when sink lower part 402 and sink upper part 404 are joined together as shown in FIG. 4B), to accommodate lens assemblies 326 of cameras 310. Sink lower part 402 further includes an elongated back groove 526, formed on lower part inner surface 506 and extending distally into proximal section 504 from a proximal end 528 of sink lower part 402. Back groove 526 together with a corresponding groove on sink upper part 404, define socket 434 (when sink lower part 402 and sink upper part 404 are joined together as shown in FIG. 4B).

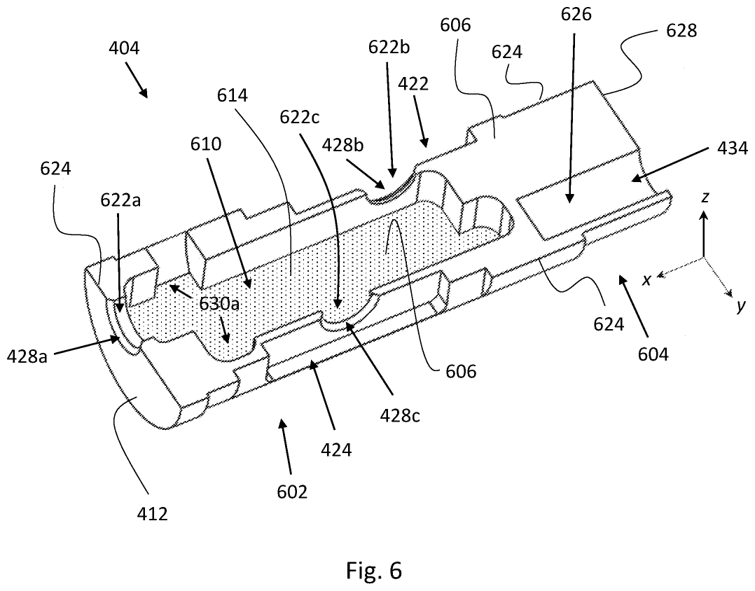

FIG. 6 schematically depicts sink upper part 404, according to some embodiments. Sink upper part 404 includes a distal section 602, a proximal section 604, an upper part inner surface 606, and an upper part outer surface 608, which as depicted in the figures may be curved, and which is positioned below upper part inner surface 606 in FIG. 6. It is noted that the orientation of sink upper part 404 has been inverted in FIG. 6 as compared to FIG. 4A, so that in FIG. 6 upper part inner surface 606 is visible while upper part outer surface 608 is mostly hidden from view, whereas in FIG. 4A upper part outer surface 608 is visible while upper part inner surface 606 is hidden from view. According to some embodiments, sink upper part 404 may generally be shaped as a half-cylinder with a depression 610 (indentation, niche, or void) formed on upper part inner surface 606. Also indicated is a depressed surface 614 of depression 610. Depression 610 extends into distal section 602 and, optionally, a distal part of proximal section 604.

Sink upper part 404 includes three grooves 622 positioned on a perimeter 624 of upper part inner surface 606: a front groove 622a, a first side-groove 622b, and a second side-groove 622c. Front groove 622a and front groove 522a (on sink lower part 402) define front hole 428a when sink lower part 402 and sink upper part 404 are joined together and aligned (i.e. with perimeters 524 and 624 overlapping, as shown in FIG. 4B) to accommodate front lens assembly 326a of front camera 310a. First side-groove 622b and first side-groove 522b define first side-hole 428b, which is configured to accommodate first side-lens assembly 326b of first side-camera 310b. Similarly, second side-groove 622c and second side-groove 522c define second side-hole 428c, which is configured to accommodate second side-lens assembly 326c of second side-camera 310c. Sink upper part 404 further includes an elongated back groove 626, formed on upper part inner surface 606 and extending distally into proximal section 604 from a proximal end 628 of sink upper part 404. Back groove 626 and back groove 526 (on sink lower part 402) define socket 434 (when sink lower part 402 and sink upper part 404 are joined together as shown in FIG. 4B).

It is noted that when sink lower part 402 and sink upper part 404 are joined together and aligned (that is, with upper part inner surface 606 of sink upper part 404 contacting lower part inner surface 506 of sink lower part 402, such that perimeter 624 overlaps with perimeter 524), depression 610 and elongated opening 510 are aligned and define a compartment (not numbered) configured to house board distal section 312 and cameras 310, as further elaborated on below. Elongated opening 510 and depression 610 are shaped such as to accommodate board distal section 312 and cameras 310 (such that board distal section 312 and cameras 310 are securely fitted in the compartment). According to some embodiments, elongated opening 510 and depression 610 may include slots or niches configured to help secure board distal section 312 and cameras 310 within the compartment. For example, as can be seen in FIG. 4A, lower front slots 530a (indicated in FIG. 5A) on the internal surface defined by elongated opening 510, and upper front slots 630a (indicated in FIG. 6) on the internal surface defined by depression 610, are configured to be aligned and to secure front foldable arm 322a there between (and thereby help secure front camera 310a). Other slots, e.g. for securing second-side foldable arm 322c are not numbered.

Making reference again to FIGS. 4A and 4B, to install optical unit 300 in sink lower part 402—that is to position optical unit 300 within/in/on sink lower part 402 as shown in FIG. 4A—optical unit 300 may be inserted via elongated opening 510 such that board distal section 312 is positioned above lower part inner surface 506 and board proximal section 314 is positioned below lower part outer surface 508. Optical unit 300 may then be turned about the longitudinal axis thereof, such that the “component side” of CBA 302 (i.e. board top surface 320) faces proximally and the “print side” of CBA 302 faces distally. Optical unit 300 may then be rotated by bringing “down” board distal section 312 into elongated opening 510, such that lens assemblies 326 rest on grooves 522, respectively (i.e. front lens assembly 326a rests on front groove 522a, and so on), and simultaneously bringing “up” board proximal section 314, such as to bring board neck member 318 into recess 518. Finally, board distal section 312 and cameras 310 may be enclosed within heat sink structure 400 by placing sink upper part 404 on top of sink lower part 402 and joining (e.g. welding) sink upper part 404 to sink lower part 402, so that board distal section 312 and cameras 310 are housed within the compartment defined by depression 610 and elongated opening 510. In particular, holes 428 secure cameras 310 and thereby also secure board distal section 312 within the compartment.

It will be understood that while in FIGS. 4A and 4B heat sink structure 400 is depicted as being formed (i.e. welded) from two parts (i.e. sink bottom part 402 and sink upper part 404), other options are possible. In particular, according to an aspect of some embodiments, there is provided heat sink structure which is integrally formed by 3D printing. The heat sink structure is similar to heat sink structure 400 but differs therefrom in not including an opening (such as elongated opening 510) with optical unit 300 being installed on the heat sink structure during the printing thereof.

While both sink lower part 402 and sink upper part 404 are depicted in the figures as being generally shaped as a half cylinder, it will be understood that other options may apply, wherein the sink lower part and the sink upper part combine to define a heat sink structure shaped as heat sink structure 400, and which allow for the insertion of optical unit 300 in a manner similar to the one described above. For example, the heat sink structure may be formed by two or more longitudinal parts, which combine to define a cylindrical shell configured to accommodate board distal section 312 and cameras 310. In particular, each of the longitudinal parts may extend along the full length of the cylindrical shell (e.g. a first part may correspond to 120 degrees of the circumference of the cylindrical shell and a second part, complementary to the first part, may correspond to 240 degrees of the circumference of the cylindrical shell). The cylindrical shell may be of different thickness at different locations along the length thereof and/or may have grooves, holes, and/or niches configured to secure board distal section 312 and cameras 310 within the compartment and for mounting illumination units 710, essentially as described above with respect to heat sink structure 400.

FIGS. 7A and 7b provide exploded and perspective views of heat sink structure 400 with illumination units 710 mounted thereon, according to some embodiments. Illumination units 710 include a front illumination unit 710a mounted on sink distal end 412, a first side-illumination unit 710b mounted within/in first side-niche 422, and a second side-illumination unit 710c mounted within/in second side-niche 424. As elaborated on below, front illumination unit 710a, first side-illumination unit 710b, and second side-illumination unit 710c are associated with front camera 310a, first side-camera 310b, and second side-camera 310c, respectively. Each of illumination units 710 includes one or more light sources, which may be mounted on a circuit board. As a non-limiting example, and as depicted in the figures, each of illumination units 710 includes a plurality of illumination modules: front illumination unit 710a may include one, two, three, or more front illumination modules 712a mounted on a front circuit board 714a, first side-illumination unit 710b may include one, two, or more illumination modules 712b mounted on a first side-circuit board 714b, and second side-illumination unit 710c may include one, two, or more illumination modules 712c (hidden from view in FIGS. 7A and 7B, but implicitly indicated by the side-window component in FIG. 11B) mounted on a second side-circuit board 714c. Each possibility corresponds to separate embodiments.

Front circuit board 714a may include a hole 716a, which is positioned and dimensioned such as to allow attaching (e.g. by gluing, brazing with tin, and/or using screws) circuit board 714a on sink distal end 412 with front lens assembly 326a fitted through (i.e. projecting through) hole 716a. According to some embodiments, and as depicted in the figures, front circuit board 714a, may be circular. Similarly, first side-circuit board 714b may include a U-shaped dent 716b, which is positioned and dimensioned such as to allow attaching (e.g. gluing, brazing with tin, and/or using screws) first side-circuit board 714b to first side-niche 422 with first side-lens assembly 236b fitted within/in dent 716b. Finally, second side-circuit board 714c may include a U-shaped dent 716c, which is positioned and dimensioned such as to allow attaching (e.g. by gluing, brazing with tin, and/or using screws) second side-circuit board 714c to second side-niche 424 with second side-lens assembly 236c fitted within/in dent 716c.

According to some embodiments, front circuit board 714a may include, on a proximal surface thereof, protruding/projecting elements adapted to fit into (i.e. be inserted into) matching notches/holes on sink distal end 412, such as to allow fastening front circuit board 714a to sink distal end 412. Similarly, first side-circuit board 714b may include protruding/projecting elements adapted to fit into matching notches/holes on first side-niche 422, such as to allow securing first-side circuit board 714b in first-side niche 422. Finally, second side-circuit board 714c may include protruding/projecting elements adapted to fit into matching notches/holes on second side-niche 424, such as to allow securing second-side circuit board 714c in second-side niche 424.

Each of illumination modules 712 includes one or more light sources, such as light emitting diodes (LEDs). As a non-limiting example, and as depicted in the figures, each of illumination modules 712 includes one, two, or more LEDs positioned adjacently to one another. According to some embodiments, each of illumination modules 712 may include a different number of LEDs (e.g. emitting light at a same wavelength and or at different wavelengths). Front illumination modules 712a are configured to illuminate a FOV of front camera 310a. First side-illumination modules 712b are configured to illuminate a FOV of first side-camera 310b. The illumination modules on second-side circuit board 714c are configured to illuminate a FOV of second side-camera 310c. In particular, a first illumination module 712a1, a second illumination module 712a2, and a third illumination module 712a3 (of front illumination modules 712a) may be positioned below, to the side of, and above front camera 310a, respectively. In one embodiment, not shown, front illumination modules 712a may be positioned such as to encircle front camera 310a. A first illumination module 712b1 and a second illumination module 712b2 (of first side-illumination modules 712b) may be positioned distally and proximally to first side-camera 310b, adjacently thereto. A first illumination module and a second illumination module (not shown) on second side-circuit board 714c may be positioned distally and proximally to second side-camera 310c, adjacently thereto.

It is noted that since first side-camera 310b and second side-camera 310c are not positioned back-to-back on board distal section 312, first side-illumination modules 712b and the illumination modules on second side-circuit board 714c may also not be positioned back-to-back. Thus, according to some embodiments, first illumination module 712b1 is not positioned back-to-back to the proximally positioned illumination module on second side-circuit board 714c and second illumination module 712b2 is not positioned back-to-back to the distally positioned illumination module on second-side-circuit board 714c.

According to some embodiments, each of illumination modules 712 is or includes a discrete light source. According to some embodiments, wherein each of illumination modules 712 includes one or more LEDs, each LED may be, for example, a white light LED, an infrared LED, a near infrared LEDs, an ultraviolet LED, and/or a combination thereof. It is noted that in embodiments wherein illumination units 710 include LEDs configured to produce light outside the visible spectrum (e.g. an infrared LED), cameras 130 will include sensors configured to detect such light (e.g. infrared light). That is, cameras 130 will have capacities of e.g. infrared cameras and so on.

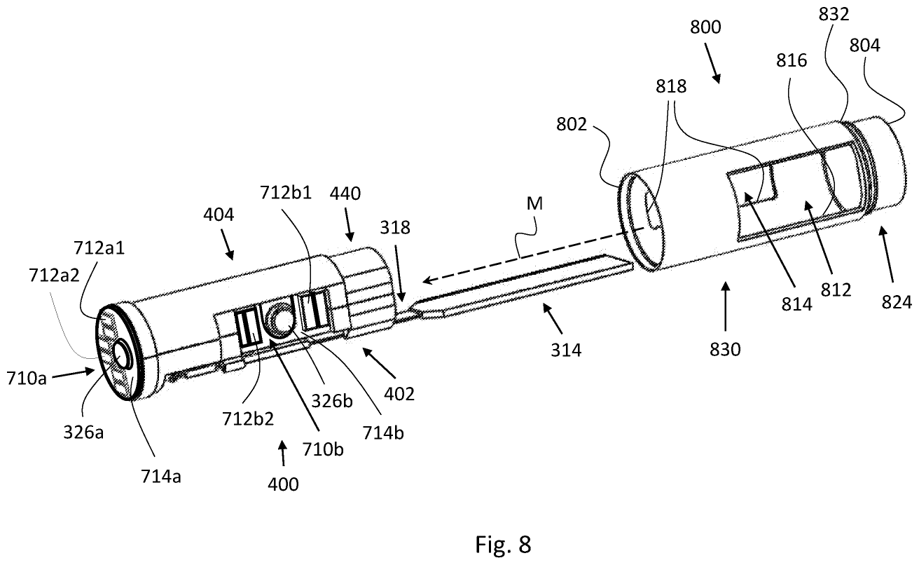

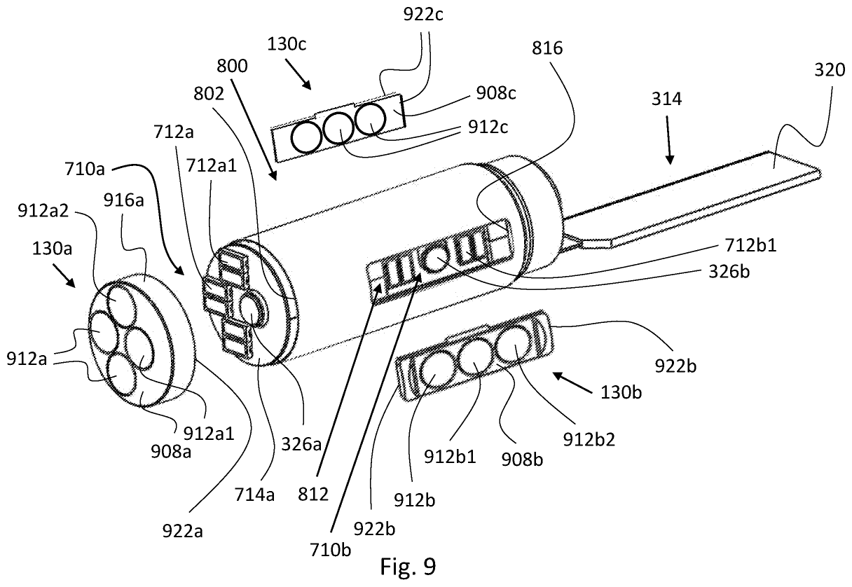

FIG. 8 schematically depicts an exploded view of heat sink structure 400 (with illumination units 710 mounted thereon) and a rigid cover 800, according to some embodiments. Cover 800 is hollow and configured (e.g. shaped and dimensioned) to be mounted on heat sink structure 400, as indicated by arrow M (and as shown in FIG. 9). In particular, in embodiments wherein heat sink structure 400 is substantially (essentially) cylindrical, cover 800 may shaped, or substantially shaped, as a cylindrical shell. According to some embodiments, cover 800 may be made of a material including stainless steel.

Cover 800 extends from a cover distal end 802 to a cover proximal end 804. Cover distal end 802 is open. According to some embodiments, cover proximal end 804 is also open, or includes one or more openings and or holes for electrical cables and a heat conduit, as elaborated on below. Cover 800 includes a first side-opening 812 and a second side-opening 814 on opposite, or essentially opposite, surfaces of cover 800. First side-opening 812 and second side-opening 814 are positioned on cover 800 such as to be adjacent to first side-niche 422 and second side-niche 424, respectively, when cover 800 is properly mounted on heat sink structure 400. Also indicated are edges 816 and edges 818 of first side-opening 812 and second side-opening 814, respectively.

According to some embodiments, a cover proximal section 824 of cover 800 may be narrower than a cover main body 830 which forms the rest of cover 800. That is, a diameter of cover main body 830 may be greater than a diameter of cover proximal section 824. Cover proximal section 824 distally extends from cover proximal end 804 until, and not including, a main body proximal end 832 (i.e. the proximal end of cover main body 830). Cover main body 830 distally extends from main body proximal end 832 to cover distal end 802.

FIG. 9 schematically depicts heat sink structure 400 with cover 800 mounted thereon (so that heat sink structure 400 is mostly hidden from view) and with window components 130 mounted on cover 800, according to some embodiments. More precisely, with respect to window components 130, the view presented in FIG. 9 is “exploded”. Window components 130 are fitted on sink distal end 412, on first side-opening 812, and on second side-opening 814, such as to protect cameras 310 and illumination units 710 (e.g. from body fluids and debris) and, at the same time, not to block, or substantially not block, the FOV of cameras 310 and not to obstruct, or substantially not to obstruct, the illumination provided by illumination modules 712 (i.e. front illumination modules 712a, first side-illumination modules 712b, and the illumination modules on circuit board 714c).

More specifically, window components 130 include a front window component 130a, a first side-window component 130b, and a second side-window component 130c.

According to some embodiments, each of window components 130 may include a respective plate with windows thereon (such that the windows are mounted within/in/on the plate in a fluid tight manner). Front window component 130a may include a front plate 908a including one or more front windows 912a. First side-window component 130b may include a first side-plate 908b including one or more first side-windows 912b. Second side-window component 130c may include a second side-plate 908c including one or more second side-windows 912c. Each of plates 908 (i.e. front plate 908a, first side-plate 908b, and second side-plate 908c) may be metallic while each of windows 912 may be transparent and may be made of glass and/or (transparent) plastic.

According to some embodiments, front window component 130a further includes a flange 916a extending proximally from a rim of plate 908a, such as to form therewith a cover. Front window component 130a is configured to be fitted (e.g. installed) on sink distal end 412, such as to encase (cover) front illumination unit 710a. The positioning of front windows 912a on plate 908a may be such that the FOV of front lens assembly 326a, and the illumination provided by front illumination modules 712a, are not blocked or substantially not blocked (e.g. by plate 908a). According to some embodiments, and as depicted in FIG. 9, front windows 912a include four windows which are positioned adjacently to front lens assembly 326a and illumination modules 712a, respectively. For example, a first window 912a1 and a second window 912a2 (from front windows 912a) may be positioned adjacently to front lens assembly 326a and first front illumination module 712a1, respectively. Front window component 130a may be affixed to cover 800 by attaching a rim 922a of flange 916a to cover distal end 802. According to some embodiments, the attachment of rim 922a to cover distal end 802 is fluid tight and may be effected, for example, by laser welding.

First side-window component 130b is fitted on first side-opening 812 such as to cover first side-illumination unit 710b (i.e. enclose first side-illumination unit 710b within/in first side-niche 422). The positioning of first side-windows 912b on first side-plate 908b may be such that the FOV of first side-lens assembly 326b, and the illumination provided by first side-illumination modules 712b, are not blocked or substantially not blocked. According to some embodiments, and as depicted in FIG. 9, first side-windows 912b include three windows positioned adjacently to lens assembly 326b and illumination modules 712b, respectively. For example, a first window 912b1 and a second window 912b2 (from first side-windows 912b) may be positioned adjacently to lens assembly 326b and first illumination module 712b1 (of first side lens-assembly 712b), respectively. According to some embodiments, first side-window component 130b may be affixed on first side-opening 812 by attaching edges 922b (not all of which are numbered) of plate 908b to edges 816 (of first side-opening 812). According to some embodiments, the attachment of edges 922b to edges 816 is fluid tight and may be effected, for example, by laser welding.

Similarly, second side-window component 130c is fitted on second side-opening 814 such as to cover second side-illumination unit 710c (i.e. enclose second side-illumination unit 710c within second side-niche 424). The positioning of second side-windows 912c on second side-plate 908c may be such that the FOV of second-side lens assembly 326c, and the illumination provided by the second side-illumination modules on second side-circuit board 714c, are not blocked or substantially not blocked. According to some embodiments, and as depicted in FIG. 9, second side-windows 912c include three windows positioned adjacently to second side-lens assembly 326c and second side-illumination modules on second side-circuit board 714c, respectively. According to some embodiments, second side-window component 130c may be affixed on second side-opening 814 by attaching edges 922c (not all of which are numbered) of second-side plate 908c to edges 818 (of second side-opening 814). According to some embodiments, the attachment of edges 922c to edges 818 is fluid tight and may be effected, for example, by laser welding.

Making reference to FIGS. 10A and 10B, shaft 102 further includes a heat conduit 1000. According to some embodiments, and as depicted in FIGS. 10A and 10B, heat conduit 1000 may be an elongated rod made of a material which is a good thermal conductor, such as copper or another metal characterized by high thermal conductivity. FIG. 10A is an exploded view of a heat sink structure 400 (with cover 800 mounted thereon) and heat conduit 1000. FIG. 10B is a perspective view of heat sink structure 400 (with cover 800 mounted thereon) and heat conduit 1000 inserted thereinto (i.e. via socket 434).

Heat conduit 1000 longitudinally extends from a conduit distal tip 1012, inserted into heat sink structure 400, to a conduit proximal tip (not shown), configured to be inserted into or positioned in handle 104. Conduit distal tip 1012 is configured to be fitted within socket 434 such as to provide high (direct) thermal coupling between heat sink structure 400 and heat conduit 1000. As heat sink structure 400 is also a good thermal conductor (at least from sink distal end 412, first side-niche 422, and second side-niche 424 to socket 434), the high thermal coupling between socket 434 and heat conduit 1000 results in a high (indirect) thermal coupling between illumination units 710 and heat conduit 1000. Thus, heat sink structure 400 and heat conduit 1000 may constitute a heat removal infrastructure which prevents overheating of shaft distal section 112 when used in an endoscopy procedure.

According to some embodiments, heat conduit 1000 is affixed in socket 434 using laser welding, brazing with tin, or the like. According to some embodiments, heat conduit 1000 is clamped between the lower part and upper part of socket 434, thereby affixing heat conduit 1000 within socket 434 without the use of bonding means.

FIGS. 11A and 11B present perspective views of shaft distal section 112 and shaft central section 114, according to some embodiments. Shaft distal section 112 includes heat sink structure 400 and cover 800 (heat sink structure 400 being hidden from view by cover 800). Further depicted is an elongated tube 1110 which may form the external part of shaft 102 at least along shaft central section 114. According to some embodiments, and as depicted in FIGS. 11A and 11B, a tube distal portion 1112 (i.e. a distal portion of tube 1110) may fitted over cover proximal section 824, as elaborated on below. However, it will be understood that other options may apply. For example, according to some embodiments, not depicted in the figures, cover 800 may be fitted over tube distal portion 1112. Heat conduit 1000 is understood to extend through elongated tube 1110 and into shaft distal section 112, and is, thus, hidden from view in FIGS. 11A and 11B.

According to some embodiments, a tube distal end 1122 may be attached to main body proximal end 832 (i.e. the proximal edge of the main body of cover 800). According to some embodiments, the attachment of tube distal end 1122 to main body proximal end 832 may be fluid tight, e.g. effected by laser welding.

It will be noted that the fluid tight attachments of tube distal end 1122 to main body proximal end 832, rim 922a of front window component 130a to cover distal end 802, edges 922b of first side-window component 130b to edges 816 on the first side-opening on cover 800, and edges 922c of second side-window component 130c to edges 818 on the second side-opening on cover 800, render shaft distal section 112 fluidly sealed. The fluid sealing of shaft distal section 112 protects electronics positioned therein from body fluids and debris (e.g. blood and tissue), and also prevents gas from entering shaft distal section 112. Preventing gas, such as air, from penetrating shaft distal section 112 is desirable as the gas may lead to corrosion of electronic components within shaft distal section 112, which may cause malfunctioning of one or more of cameras 310 and illumination modules 712. In addition, gas present within shaft distal section 112 may undergo condensation therein, e.g. forming a condensate on the internal surfaces of windows 912 (i.e. front windows 912a, first side-windows 912b, and second side-windows 912c), thereby blurring the video images provided by cameras 130. The fluid sealing of shaft distal section 112 is further configured to be durable under high pressures, such as to allow shaft 102 to undergo sterilization procedures (e.g. autoclave sterilization).

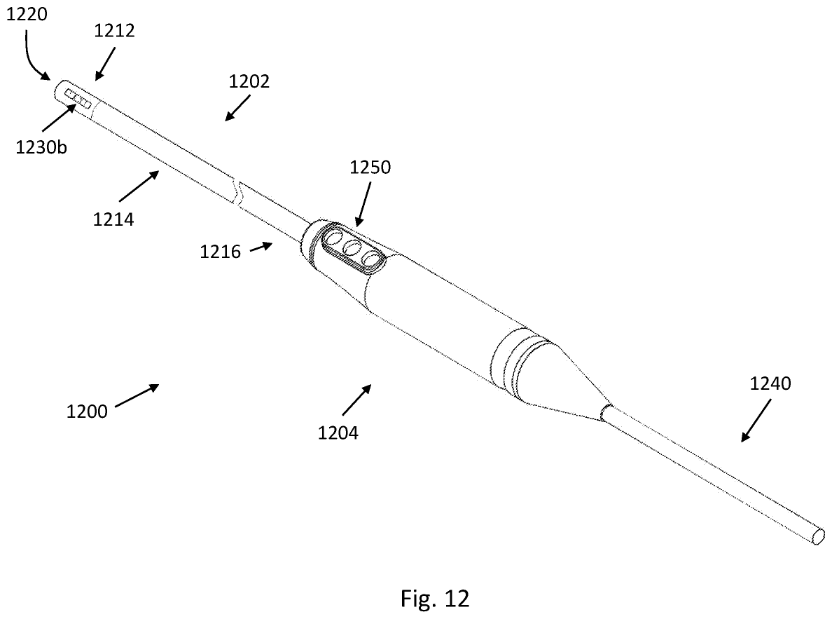

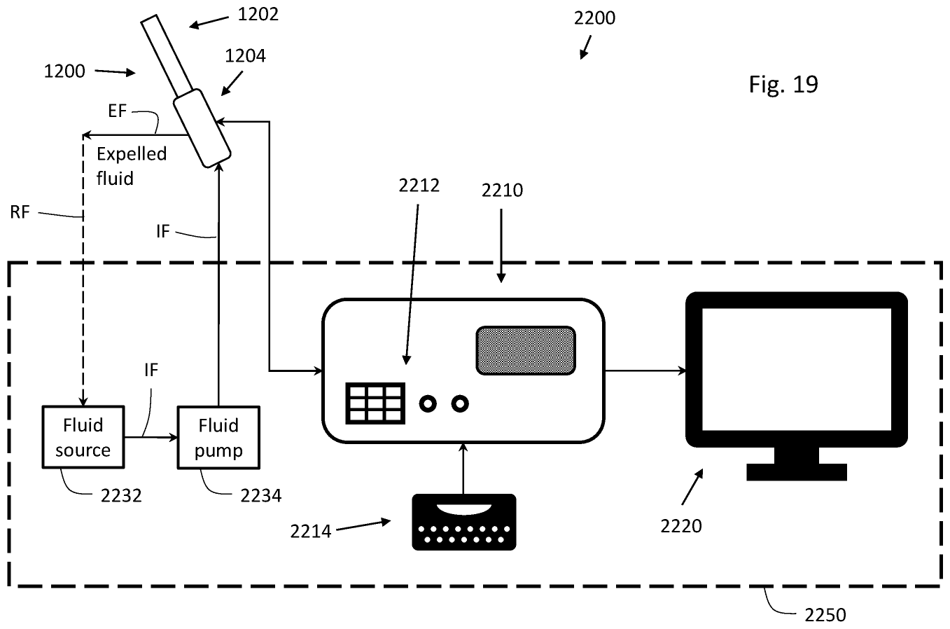

According to an aspect of some embodiments, schematically depicted in FIGS. 12-18, there is provided an endoscope. FIG. 12 schematically depicts such an endoscope, an endoscope 1200, according to some embodiments. FIG. 19 schematically depicts an image monitoring system including endoscope 1200.

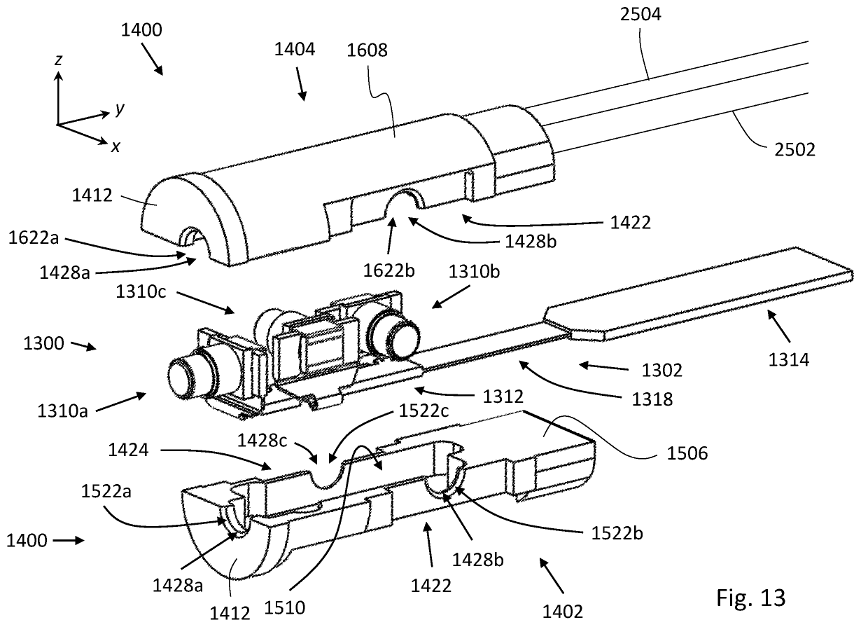

According to some embodiments, and as depicted in FIG. 12, endoscope 1200 is a rigid endoscope. According to some alternative embodiments, not depicted in the figures, endoscope 1200 may be a semi-rigid endoscope, as elaborated on below. Endoscope 1200 is similar to endoscope 100 (of FIG. 1) but differs therefrom at least in including two (or more) conduits (depicted in FIG. 13), whereas endoscope 100 includes a single conduit, i.e. heat conduit 1000 (of FIGS. 10A and 10B). According to some embodiments, the two conduits form part of an “active” heat removal infrastructure. More specifically, the two conduits may be hollow (i.e. the two conduits may be tubes or ducts) and configured for fluid conveyance, for example, cooled gas circulation, such as to enhance the rate of heat removal from a distal section of endoscope 1200. The heat removal infrastructure is thus said to be “active” in the sense of involving, e.g. pumping and/or suction of fluids (i.e. liquid and/or gas), as elaborated on below. Alternatively, according to some embodiments, the two conduits may each be similar to heat conduit 1000 of endoscope 100.

Endoscope 1200 includes an elongated shaft 1202, configured to be inserted into an anatomical site, and a handle 1204, configured to be held by a user of endoscope 1200 and to facilitate guidance and manipulation of shaft 1202 within the anatomical site. Shaft 1202 is mounted on handle 1204. According to some embodiments, shaft 1202 is integrated in endoscope 1200 as an extension of handle 1204. According to some embodiments, shaft 1202 is configured to be detachably mountable on handle 1204. Also indicated is a utility cable 1240, the function of which is described below.

According to some embodiments, shaft 1202 and handle 1204 may be similar to shaft 102 and handle 104 (of endoscope 100) up to differences resulting from the form and function of the two conduits (of endoscope 1200). Thus, in the description of endoscope 1200, features shared in common with endoscope 100, may be described in significantly less detail or not enumerated at all.

It is noted that while in the depictions provided by FIG. 1 and FIG. 12 handles 104 and 1204 are portrayed as differing in shape, this need not necessarily be the case. In particular, while in FIG. 1 handle 104 is shaped similarly to a handle of a pistol, according to some alternative embodiments, handle 104 may be shaped essentially as a linear rod (whose diameter may change along the length thereof). Similarly, while in FIG. 12 handle 1204 is shaped essentially as a linear rod, according to some alternative embodiments, handle 1204 may be shaped similarly to a handle of a pistol.

Shaft 1202 includes a shaft distal section 1212, a shaft central section 1214, a shaft proximal section 1216, and a shaft distal tip 1220 (whereat shaft distal section 1212 terminates in the distal direction).

Shaft distal section 1212 houses at least three cameras 1310 (shown in FIG. 13) positioned behind window components 1230 and hidden from view thereby in FIG. 12. Cameras 1310 and window components 1230 may be similar to cameras 310 and window components 130 of endoscope 100. Shaft distal section 1212 further includes one or more illumination units (not shown), associated with cameras 1310, positioned behind window components 1230, and hidden from view thereby. The illumination units may be similar to illumination units 710 of endoscope 100 and are configured to illuminate the FOV of cameras 1310, essentially as described above with respect to cameras 310 and illumination units 710 of endoscope 100.

Handle 1204 may include a user control interface 1250 including one or more buttons, knobs, switches, a touch panel, and/or the like, and configured to allow a user to control endoscope 1200 functions. User control interface 1250 is functionally associated with cameras 1310 and the illumination units in shaft distal section 1212. According to some embodiments, the user control interface may allow, for example, to control zoom, focus, record/stop recording, and/or freeze frame functions of cameras 1310 and/or to adjust the intensity of light provided by the illumination units collectively and/or individually.

According to some embodiments, wherein endoscope 1200 includes an active heat removal infrastructure, user control interface 1250 may further be configured to control functions of the active heat removal infrastructure, as elaborated on below.

According to some embodiments, endoscope 1200 may be (i) directly maneuvered by a user through the manipulation of handle 1204, as well as (ii) indirectly maneuvered, via robotics, e.g. using a robotic arm or other suitable gripping means configured to allow manipulation of handle 1204.

FIG. 13 is a schematic, exploded view of a heat sink structure 1400 with (i) a pair of conduits, a first conduit 2502 and a second conduit 2504, inserted thereinto, and (ii) an optical unit 1300 accommodated in heat sink structure 1400, according to some embodiments. Heat sink structure 1400 is configured to be housed in shaft distal section 1212. As elaborated on below, heat sink structure 1400 differs from heat sink structure 400 (of endoscope 100) in being configured to have inserted thereinto a pair of conduits (i.e. conduits 2502 and 2504), instead of a single conduit, but may otherwise be essentially similar to heat sink structure 400.

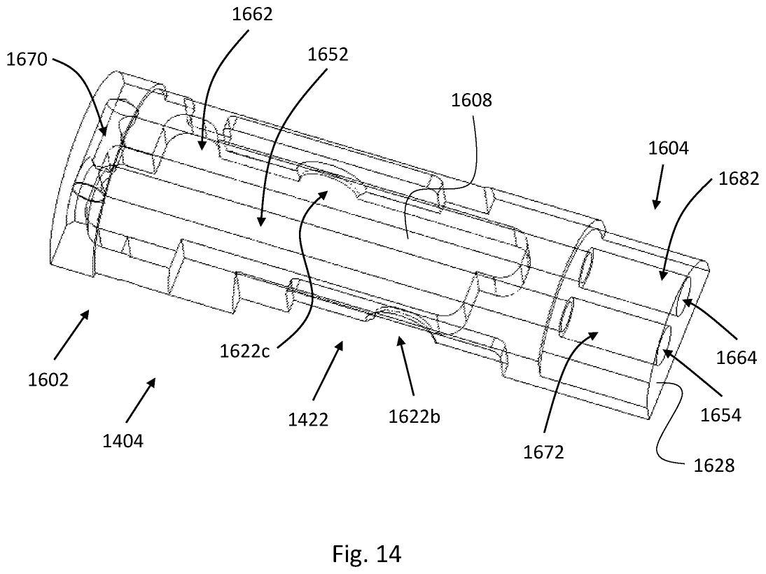

More specifically, heat sink structure 1400 is configured to accommodate cameras 1310 and to have mounted thereon the illumination units. Heat sink structure 1400 is further configured to absorb heat generated primarily by the illumination units, and to expel the heat via first conduit 2502 and/or second conduit 2504, as explained below. According to some embodiments, heat sink structure 1400 includes a sink lower part 1402 and a sink upper part 1404, which are joined onto one another, e.g. by welding and/or gluing and/or using screws. According to some embodiments, sink lower part 1402 may differ from sink lower part 402 (of heat sink structure 400) in not including a back groove (such as back groove 526) but may otherwise be essentially similar thereto. Sink upper part 1404 may differ from sink upper part 404 (of heat sink structure 400), as elaborated on below in the description of FIGS. 14-16B.

Optical unit 1300 includes cameras 1310 (e.g. a front camera 1310a and two side-cameras 1310b and 1310c, which may be positioned not back-to-back) and may be similar to optical unit 300 (of endoscope 100). Also indicated are a circuit board assembly 1302 (CBA; e.g. a printed circuit board and electronic components, such as processors, amplifiers, and discrete components), a board distal section 1312 and a board proximal section 1314 (of CBA 1302), and a board neck member 1318, which may be similar to CBA 302, board distal section 312, board proximal section 314, and board neck member 318 (of optical unit 300), respectively.

Further indicated are a first side-niche 1422 and a second side-niche 1424, which may be similar to first side-niche 422 and second side-niche 424 of heat sink structure 400. First side-niche 1422 and second side-niche 1424 are configured to have mounted therein/thereon illumination units (not shown), such as, or similar to, first side-illumination unit 710b and second side-illumination unit 710c. Also indicated are a front groove 1522a on sink lower part 1402 and a front groove 1622a on sink upper part 1404, which together define a front hole 1428a for a lens assembly of front camera 1310a. Similarly, a first side-hole 1428b for a lens assembly of first side-camera 1310b, within first side-niche 1422, is defined by a first side-groove 1522b on sink lower part 1402 and a first side-groove 1622b on sink upper part 1404. Finally, a second side-hole 1428c for a lens assembly of second side-camera 1310c, within second side-niche 1424, is defined by a second side-groove 1522c on sink lower part 1402 and a second side-groove 1622c (shown in FIG. 14) on sink upper part 1404. Holes 1428a, 1428b, and 1428c may be similar to holes 428a, 428b, and 428c of heat sink structure 400.

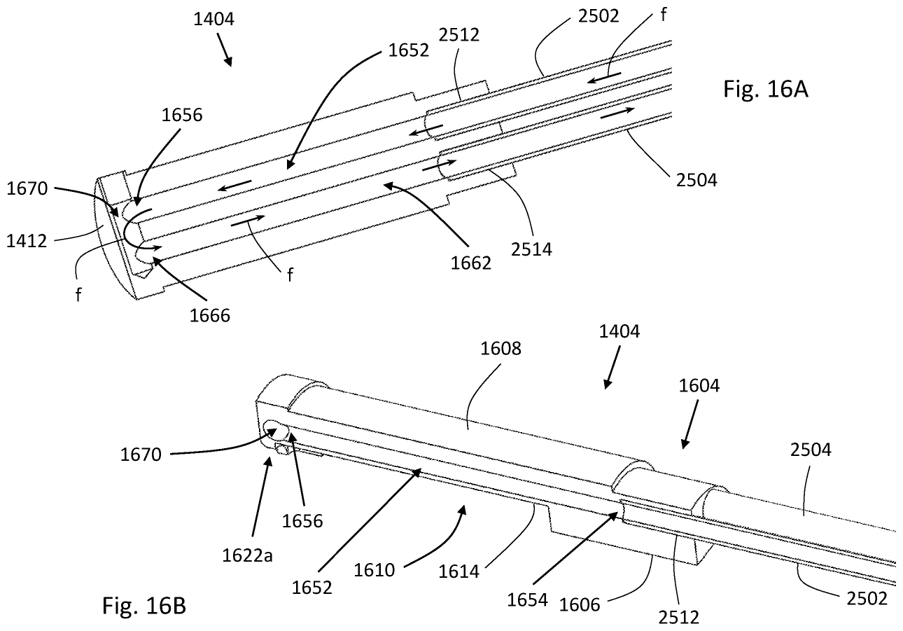

FIG. 14 is a schematic, perspective, semi-transparent view of sink upper part 1404, according to some embodiments. Sink upper part 1404 includes a distal section 1602, a proximal section 1604, an upper part inner surface 1606 (indicated in FIG. 16B), and an upper part outer surface 1608, which, as depicted in the figures, may be curved. According to some embodiments, sink upper part 1404 may generally be shaped as a half-cylinder with a depression 1610 (indicated in FIG. 16B) formed on upper part inner surface 1606. Depression 1610 extends into distal section 1602 and, optionally, a distal part of proximal section 1604.

A first channel 1652 extends from a first channel proximal end 1654 to a first channel distal end 1656 (indicated in FIG. 16A). A second channel 1662 extends from a second channel proximal end 1664 to a second channel distal end 1666 (indicated in FIG. 16A). More specifically, first channel 1652 and second channel 1662, extend distally into sink upper part 1404 from a proximal end 1628 thereof. According to some embodiments, first channel 1652 and second channel 1662 may extend in parallel to a longitudinal axis of sink upper part 1404 (and in parallel to one another). First conduit 2502 and second conduit 2504 are configured to be inserted into first channel 1652 and second channel 1662 as elaborated on below. According to some embodiments, a front channel 1670 fluidly couples first channel distal end 1656 to second channel distal end 1666. According to some embodiments, front channel 1670 may be positioned (e.g. embedded) within distal section 1602 (of sink upper part 1404), so that first channel 1652 and second channel 1662 each extends substantially along the full length of sink upper part 1404. According to some embodiments, front channel 1670 may extend perpendicularly to the longitudinal axis of sink upper part 1404.

According to some embodiments, channels 1652 and 1662 may be embedded within sink upper part 1404. That is, channels 1652 and 1662 may form elongated passages within sink upper part 1404. According to such some embodiments, due to the presence of channels 1652 and 1662 (which extend within sink upper part 1404 between a depressed surface 1614 (indicated in FIG. 16B) of depression 1610 and upper part outer surface 1608), depression 1610 may be shallower than depression 610 (shown in FIG. 6) of heat sink structure 400. According to some embodiments, channels 1652 and 1662 may be characterized by a diameter measuring between about 1 millimeter and about 5 millimeters.

According to some alternative embodiments, channels 1652 and 1662 do not distally extend beyond proximal section 1604. According to some such embodiments, front channel 1670 may also be positioned in proximal section 1604. Alternatively, according to some embodiments (not depicted in the figures), wherein the heat removal infrastructure is configured to convey gas, particularly a noble gas, heat sink structure 1400 does not include a channel (such as front channel 1670) that connects first channel 1652 to second channel 1662. Instead, each of channels 1652 and 1662 (distally) terminates at the compartment defined by an elongated opening 1510 (described below) in sink lower part 1402 and depression 1610. Thus, for example, cooling gas pumped into first channel 1652, enters the compartment via a distal end of first channel 1652, and exits the compartment, into second channel 1662, via a distal end of second channel 1662.

Alternatively, according to some embodiments not depicted in the figures, one or both of channels 1652 and 1662 may be partially or fully exposed within depression 1610. For example, between a proximal wall and a distal wall (not numbered) of depression 1610, channels 1652 and 1662 may be defined by respective tubes which extend between the proximal wall and the distal wall of depression 1610.

A first channel proximal portion 1672 extends distally from first channel proximal end 1654. Similarly, a second channel proximal portion 1682 extends proximally from second channel proximal end 1664. First channel proximal portion 1672 and second channel proximal portions 1682 are configured to have fitted thereinto first conduit 2502 and second conduit 2504, respectively. More specifically, first channel proximal portion 1672 may be configured (e.g. dimensioned) to have fitted thereinto a first conduit distal portion 2512, such as to fluidly connect first conduit 2502 and first channel 1652. Similarly, second channel proximal portion 1682 may be configured to have fitted thereinto a second conduit distal portion 2514, such as to fluidly connect second conduit 2504 and second channel 1662. Moreover, the fitting of first conduit distal portion 2512 within first channel proximal portion 1672 may be such as to provide high (direct) thermal coupling between sink upper part 1404 and first conduit 2502. Similarly, the fitting of second conduit distal portion 2514 within second channel proximal portion 1682 may be such as to provide high (direct) thermal coupling between sink upper part 1404 and second conduit 2504.

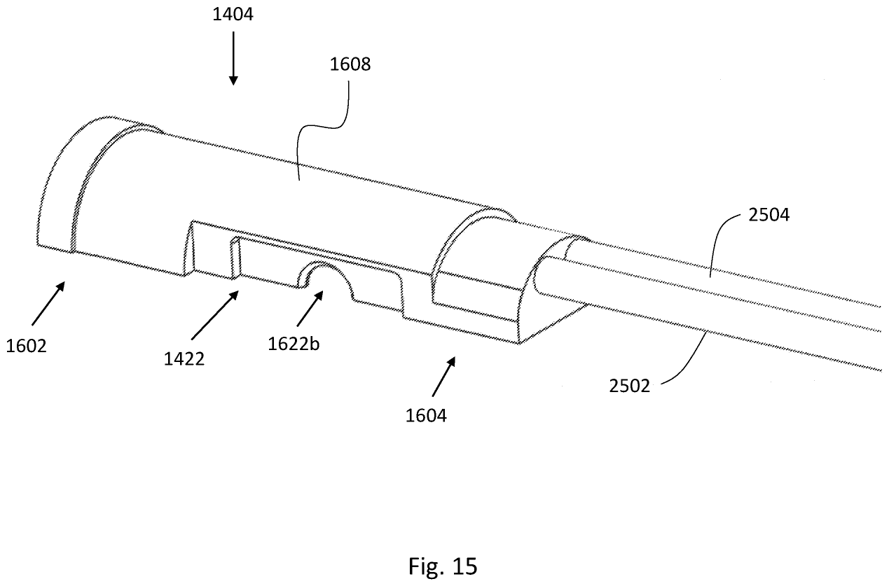

FIG. 15 presents a schematic, perspective view of sink upper part 1404 with first conduit 2502 and second conduit 2504 inserted into sink upper part 1404, according to some embodiments. More specifically, sink upper part 1404 is shown with first conduit distal portion 2512 (i.e. the distal portion of first conduit 2502) and second conduit distal portion 2514 (i.e. the distal portion of second conduit 2504) inserted into sink upper part 1404. According to some embodiments, first conduit distal portion 2512 may be affixed within first channel 1652 in proximal section 1604, for example, by welding, heat bonding, and/or gluing. Similarly, according to some embodiments, second conduit distal portion 2514 may be affixed within second channel 1662 in proximal section 1604, for example, by welding, heat bonding, and/or gluing. According to some embodiments, first conduit 2502 and second conduit 2504 may each be integrated into sink upper part 1404.

FIG. 16A presents a perspective cross-sectional view of sink upper part 1404, according to some embodiments. The cross-section “cuts” along a plane which is parallel to the xy-plane (as defined in FIG. 13) and which halves each of conduits 2502 and 2504 along the respective lengths thereof. Arrows f indicate direction of flow of fluid pumped into second conduit 2504 via a proximal end thereof.

FIG. 16B presents a perspective cross-sectional view of sink upper part 1404, according to some embodiments. The cross-section “cuts” along a plane which is parallel to the yz-plane (as defined in FIG. 13) and which halves first conduit 2502 (and, in particular, first conduit distal portion 2512) along the length thereof.

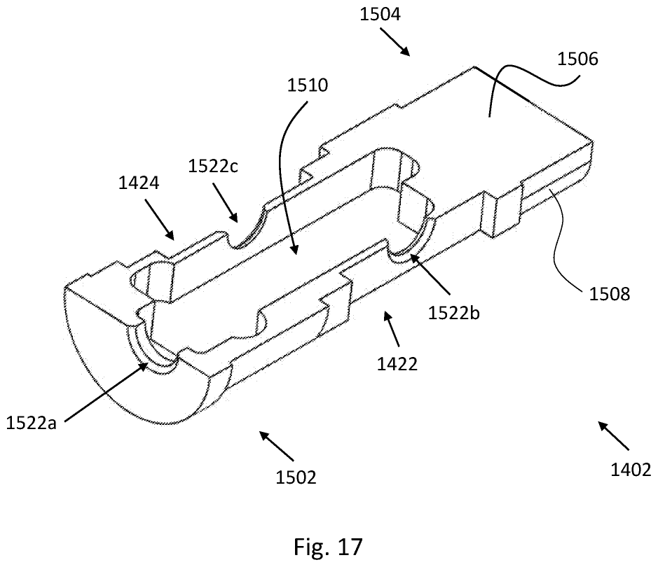

FIG. 17 presents a schematic, perspective view of sink lower part 1402, according to some embodiments. Sink lower part 1402 may be similar to sink lower part 402 (shown in FIG. 5A) of endoscope 100, potentially differing therefrom as elaborated on below. Sink lower part 1402 includes a distal section 1502 and a proximal section 1504. Proximal section 1504 includes a lower part inner surface 1506, and a lower part outer surface 1508, which may be curved. Sink lower part 1402 further includes elongated opening 1510, which extends in the longitudinal direction and is similar to elongated opening 510 in sink lower part 402.

Similarly to lower part outer surface 508 of sink lower part 402, lower part outer surface 1508 is longitudinally indented along proximal section 1504, such as to form a recess (hidden from view in the figures) similar to recess 518 (of sink lower part 402). The recess is configured to accommodate board neck member 1318, and to restrict both lateral and vertical motion thereof, thereby helping to secure board distal section 1312 and cameras 1310 within heat sink structure 1400. According to some embodiments, and as depicted in the figures, first conduit 2502 and second conduit 2504 extend only into sink upper part 1404 but not into sink lower part 1402. According to some such embodiments, lower part inner surface 1506 may be fully flat, or substantially fully flat, along proximal section 1504, in order to maximize the thermal coupling between lower part inner surface 1506 (of sink lower part 1402) and upper part inner surface 1606 (of sink upper part 1404), and thereby increase the thermal coupling between sink lower part 1402 and conduits 2502 and 2504. In particular, unlike proximal section 504 of endoscope 100, proximal section 1504 need not include a groove.

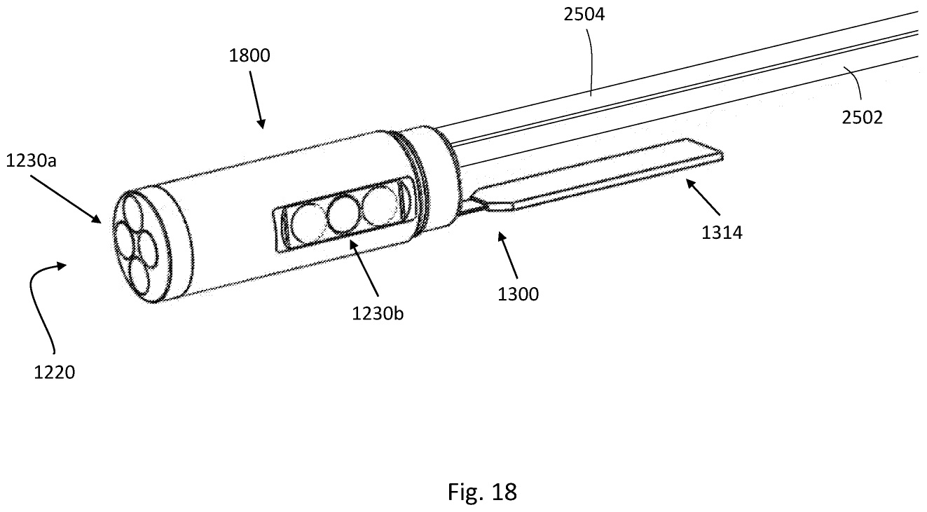

FIG. 18 is a perspective view of heat sink structure 1400 with a cover 1800 mounted thereon, and conduits 2502 and 2504 inserted thereinto (i.e. into channels 1652 and 1662, respectively). Also indicated are window components 1230a and 1230b.

Making reference again to FIGS. 14-16B, channels 1652 and 1662 are configured for circulation/conveyance of a cooling fluid. More specifically (and as indicated by arrows fin FIG. 16A), according to some embodiments, endoscope 1200 may be configured such that cooling fluid (cool/cold gas or liquid) pumped into first conduit 2502: (i) flows distally along first conduit 2502, exiting therefrom into first channel 1652, via first conduit proximal portion 2512; (ii) exits first channel 1652, into front channel 1670, via first channel distal end 1656; (iii) flows along front channel 1670 and enters into second channel 1662 via second channel distal end 1666; (iv) flows in the proximal direction along second channel 1662; and (v) exits heat sink structure 1400, into second conduit 2504, via second conduit distal portion 2514.

Additionally/alternatively, according to some embodiments, endoscope 1200 may be configured such as to allow circulation of fluid in an opposite sense to that indicated by arrows f. That is, endoscope 1200 may be configured such as to allow pumping of cooling fluid into heat sink structure 1400 via second conduit 2504 and removal therefrom via first conduit 2502 (so that the cooling fluid enters heat sink structure 1400 via second channel 1662 and exits heat sink structure 1400 via first channel 1652).

As the cooling fluid circulates through heat sink structure 1400, the cooling fluid absorbs, through walls of channels 1652 and 1662 and front channel 1670, heat generated by electronic components in heat sink structure 1400, and, in particular, by LEDs in the illumination units. More specifically, as the cooling fluid (distally) flows through first channel 1652, the cooling fluid may absorb heat generated by the illumination unit mounted in first side-niche 1422. As the cooling fluid flows through front channel 1670, the cooling fluid may absorb heat generated by the (front) illumination unit mounted on a sink distal end 1412 (indicated in FIG. 13). As the cooling fluid (proximally) flows through second channel 1662, the cooling fluid may absorb heat generated by the illumination unit mounted in second side-niche 1424. Thus, cooling fluid flowing through second conduit 2504 may be of higher temperature than cooling fluid flowing through first conduit 2502, having absorbed heat from heat sink structure 1400 when flowing therethrough.

According to some embodiments, first conduit 2502 may be thermally insulating (e.g. made of a thermally insulating material), such as to prevent absorption of heat by cooling fluid flowing therethrough towards heat sink structure 1400. In particular, first conduit 2502 may be thermally insulated from second conduit 2504 such as to prevent transfer of heat from cooling fluid in second conduit 2504 to cooling fluid in first conduit 2502 (which may be of lower temperature than the cooling fluid in second conduit 2504). According to some embodiments, both first conduit 2502 and second conduit 2504 may be thermally insulating.

According to some embodiments, handle 1204 may include a fluid reservoir (e.g. a fluid container, not shown) and a pump (not shown) for circulating the cooling fluid as described above. The fluid reservoir is fluidly coupled to the pump and to conduits 2502 and 2504. The pump is configured to force fluid from the fluid reservoir into e.g. first conduit 2502, thereby causing the fluid to circulate through heat sink structure 1400, i.e. via channels 1652 and 1662 (and front channel 1670). On exiting heat sink structure 1400, the fluid is forced back, via second conduit 2504, into the fluid reservoir, and so on. The fluid reservoir, the pump, conduits 2502 and 2504, and channels 1652 and 1662, (as well as front channel 1670 and any other tubes/ducts, e.g. connecting the fluid reservoir to the pump), thus constitute a self-contained (cooling) fluid-circulation system, in the sense of being fully included in endoscope 1200 (at least up to a power source for circulating the cooling fluid). In particular, according to some embodiments, the self-contained fluid-circulation system may constitute a closed fluid-circulation system in the sense that during operation of endoscope 1200, none of the circulated fluid is expelled and neither is new (external) fluid added.

According to some embodiments, handle 1204 or shaft 1202 may include a one-way valve configured to prevent backflow of the cooling fluid.

The fluid reservoir may be configured for rapid expulsion of heat to the environment of handle 1204, i.e. to the ambient air. More specifically, the fluid reservoir may be positioned in/on handle 1204 such as to facilitate rapid heat dissipation (i.e. rapid heat transfer from endoscope 1200, and, in particular, handle 1204, to the environment thereof). For example, according to some embodiments, one or more walls of the fluid reservoir may be exposed to the ambient air. The walls may be directly exposed, e.g. forming a part of the surface of handle 1204, or indirectly exposed, being positioned below a protective grating on the surface of handle 1204. The exposed walls may be made of, or include, a material characterized by high thermal conductivity, thereby ensuring rapid heat dissipation.

According to some embodiments, handle 1204 may include a port (not shown), allowing to fill and empty the fluid reservoir (thereby allowing to replace the cooling fluid).

According to some embodiments, the fluid reservoir may be removably installed in/on handle 1204, thereby allowing to replace the cooling fluid by removing the fluid reservoir (and filling the fluid reservoir with fresh and cold cooling fluid) and/or even by replacing the fluid reservoir (e.g. the fluid reservoir may constitute a removable capsule).

According to some embodiments, the cooling fluid may be water, air, CO2, nitrogen, or ammonia.

According to some embodiments, the pump may be powered by a power source, such as a removable and/or rechargeable battery, included in handle 1204. According to some embodiments, the pump may be powered by an external power source, coupled thereto via utility cable 1240 (shown in FIG. 12). User control interface 1250 may be configured to allow a user to control the fluid-circulation system (e.g. to turn on/off the fluid-circulation system). In particular, according to some embodiments, the fluid-circulation system may include features controllable by user control interface 1250, such as a rate of the fluid circulation (which in turn may control the rate of heat removal).

According to some embodiments, in particular embodiments wherein both first conduit 2502 and second conduit 2504 are thermally insulating, the pump may be configured to allow reversing the direction of flow of the cooling fluid.

According to some embodiments, endoscope 1200 may include a thermometer (not shown) configured to measure the temperature of the cooling fluid exiting shaft distal section 1212. According to some embodiments, the thermometer may be positioned along second conduit 2504. According to some embodiments, the thermometer may be positioned in handle 1204. According to some embodiments, the user control interface (in handle 1204) may be configured to display the temperature of the cooling fluid exiting shaft distal section 1212. According to some embodiments, the user control interface may be configured to display a warning, when the temperature exceeds a threshold. According to such some embodiments, endoscope 1200 may further include an additional thermometer (not shown) configured to measure the temperature of the cooling fluid flowing into shaft distal section 1212. According to some embodiments, the additional thermometer may be positioned along first conduit 2502. According to some embodiments, the additional thermometer may be positioned in handle 1204. According to some embodiments, the user control interface may be configured to display the temperature of the cooling fluid entering shaft distal section 1212. According to some embodiments, the user control interface may be configured to display a warning when the difference in the measured temperatures (the difference between the temperature of the exiting cooling fluid and the temperature of the entering cooling fluid) exceeds a threshold.

According to some embodiments, endoscope 1200 may be connected to a main control unit (not shown), e.g. via a utility cable. Additionally/alternatively, endoscope 1200 may include a wireless communication unit (e.g. a Bluetooth antenna) configured to communicatively associate endoscope 1200 with the main control unit. The main control unit may be similar to main control unit 210 of medical imaging system 200, optionally differing therefrom in being configured to (i) allow controlling features related to the fluid-circulation system, e.g. via a user control interface included in main control unit and/or associated input devices, and in being configured to power the fluid-circulation system. The user control interface may further be configured to present information relating to the operation of the fluid-circulation. Additionally/alternatively, such information may be presented on a monitor connected to the main control unit.

Alternatively, according to some embodiments, endoscope 1200 does not include a fluid reservoir. Optionally, according to some such embodiments, endoscope 1200 does not include a pump. Such embodiments constitute fluid circulation systems, which are not “self-contained” in endoscope 1200 (in the sense that some components of the fluid-circulation system are not housed in endoscope 1200). In such embodiments, handle 1204 may include infrastructure (not shown) for receiving cooling fluid from an external fluid source and for expelling the cooling fluid after the cooling fluid has been circulated through heat sink structure 1400. In particular, handle 1204 may include a first port (not shown) for the intake of cooling fluid (e.g. from a fluid delivery pipe), and a second port (not shown) for expelling the cooling fluid from endoscope 1200 (e.g. via a fluid expulsion pipe). The first port may be fluidly connected to first conduit 2502. In such embodiments, first conduit 2502 may extend into handle 1204 until the first port. Alternatively, according to some embodiments, the first port may be fluidly coupled to first conduit 2502 via a first cooling fluid tube in handle 1204 (i.e. a tube included in handle 1204 and configured for conveying the cooling fluid). The second port may be fluidly connected to second conduit 2504. In such embodiments, second conduit 2504 may extend into handle 1204 until the second port. Alternatively, according to some embodiments, the second port may be fluidly coupled to second conduit 2504 via a second cooling fluid tube in handle 1204. (Alternatively, according to some embodiments, the first port may be fluidly connected, or fluidly coupled, to second conduit 2504, and the second port may be fluidly connected, or fluidly coupled, to first conduit 2502.)

According to some embodiments, the first port may be configured to prevent exit of fluid therethrough and/or the second port may be configured to prevent entry of fluid therethrough. According to some embodiments, the fluid delivery pipe and the first port, and/or the second port and the fluid expulsion pipe, are each fluidly coupled via leak-free connection, such as a luer connection mechanism. According to some embodiments, the fluid delivery pipe may include (on a distal end thereof) a hanger plug configured to be connected to a matching hanger socket in the first port. According to some such embodiments, the hanger plug may be integrally formed on a cap portion of the fluid delivery pipe or on a distributor disc of the fluid delivery pipe.

According to some embodiments in which endoscope 1200 includes a self-contained fluid-circulation system, endoscope 1200 may be used in conjunction with a main control unit, a monitor, and, optionally, one or more input devices, to provide a medical imaging system. The medical imaging system may be similar to medical imaging system 200, but may differ therefrom not only in including endoscope 1200 in place of endoscope 100, but also in that the main control unit may be configured to monitor and optionally control functions of endoscope 1200 related to the fluid-circulation (such as to begin/stop circulating fluid, increase the rate of fluid circulation, and so on), as described above.

FIG. 19 schematically depicts a medical imaging system 2200 including endoscope 1200, according to some embodiments in which endoscope 1200 does not include a self-contained fluid-circulation system. In addition to endoscope 1200, medical imaging system 2200 includes a main control unit 2210, a monitor 2220, a fluid source 2232, and a pump 2234. Main control unit 2210 may be similar to main control unit 210 of medical imaging system 200, differing therefrom in including additional features/components related to the fluid-circulation system. Monitor 2220 may be essentially similar to monitor 220 of medical imaging system 200.

Also indicated are a user control interface 2212 and one or more input devices 2214, which may be similar to user control interface 212 and input devices 214 of medical imaging system 200.

According to some embodiments, endoscope 1200 may be connected to main control unit 2210 via utility cable 1240. Additionally/alternatively, endoscope 1200 may include a wireless communication unit configured to communicatively associate endoscope 1200 with main control unit 2210.

According to some embodiments, fluid source 2232 may be a fluid tank. Alternatively, according to some embodiments, fluid source 2232 may be a common source of fluid, such as a hospital water (or other type of liquid) supply system or a (hospital) medical gas supply system. Fluid source 2232 is fluidly coupled to pump 2234, which, in turn, is fluidly coupled to endoscope 1200, e.g. via the first port. During endoscope 1200 operation, fluid (from fluid source 2232) may be injected into handle 1204 using pump 2234 (as indicated by arrows IF). After circulating through heat sink structure 1400, the fluid is expelled from handle 1204 (as indicated by an arrow EF).

A fluid-intake tube (not shown) may be connected to the first port (in handle 1204), on one end thereof, and fluidly coupled to pump 2234 via a second end thereof. A fluid-expulsion tube (not shown) may be connected to the second port (in handle 1204).

According to some embodiments, wherein the pumped (cooling) fluid is a gas such as air (e.g. cooled or cold air), after being pumped into endoscope 1200 and circulated therethrough, the air may be released to the environment (in which case the second port may include a one-way valve which prevents outside air from entering and allows for gas release). According to some embodiments, wherein the pumped (cooling) fluid is a liquid such as water (e.g. cooled or cold water), after being pumped into endoscope 1200 and circulated therethrough, the liquid may be expelled into a drain (e.g. a hospital sewage system) or a container for subsequent disposal. It is noted that in the last two embodiments, medical imaging system 2200 is configured for “open” fluid-circulation in the sense that the expelled fluid is not recirculated by is instead replaced by “new” (and cold) fluid.

According to some embodiments and as depicted in FIG. 19, main control unit 2210, monitor 2220, pump 2234, and optionally, fluid source 2232, may be housed in a single common housing, which functions as a medical imaging station 2250 (indicated by a dashed box). According to some embodiments, medical imaging station 2250 may be mobile (e.g. mounted on wheels).

According to some embodiments, fluid source 2232 is, or may function as, a fluid reservoir. That is, the fluid expelled from handle 1204 (indicated by arrow EF) is recirculated (directed back) into fluid source 2232 (as indicated by a dashed arrow RF). It is noted that in such embodiments, medical imaging system 2200 is configured to provide “closed” fluid-circulation. According to some embodiments, the fluid source 2232 may be configured to release heat to the environment and/or may be actively cooled.

According to some embodiments, wherein medical imaging system 2200 is configured to provide closed fluid circulation, medical imaging system 2200 may further be configured to actively cool the cooling fluid in a similar manner to an air conditioner, with medical imaging system 2200 further including a condenser (not shown). In such embodiments, the cooling fluid pumped into endoscope 1200 may be a liquid, which may evaporate when passing through shaft distal section 1212. The cooling fluid may exit endoscope 1200 as a gas, which is next conveyed to the condenser, whereat the gas is condensed into liquid, and heat collected by the cooling fluid, in passage through shaft distal section 1212, is released to the environment.

According to some embodiments, pump 2234 may be configured to reverse the direction of flow of the cooling fluid.

It is to be understood that the scope of the disclosure also covers shafts for semi-rigid endoscopes. As used herein, according to some embodiments, a “semi-rigid endoscope” may refer to an endoscope including a semi-rigid shaft. The semi-rigid shaft may include an elongated rigid member, a distal tip portion, and a maneuvering portion mounted between, and mechanically coupling, the rigid member and the distal tip portion, as described in PCT application, publication No. WO2016181404, to A. Levy, which is incorporated herein by reference in its entirety. The semi-rigid shaft includes at least two cameras: a front camera and one or more side-cameras. The front camera is positioned on the distal tip portion. Each of the one or more side-cameras may be positioned on the distal tip portion, the maneuvering portion, or the elongated member. The semi-rigid shaft further includes one or more illumination components configured to illuminate the FOV provided by the at least two cameras. The maneuvering portion is configured to bend, rotate, and/or angulate the distal tip portion, and thereby controllably change the combined FOV provided by the at least two cameras.

Thus, according to an aspect of some embodiments, not depicted in the figures, there is provided a semi-rigid endoscope. The semi-rigid endoscope may be similar to endoscope 100 or endoscope 1200, but differs therefrom in including a semi-rigid shaft, as described in the preceding paragraph, instead of a rigid shaft. In particular, the at least two cameras and illumination units of the semi-rigid endoscope may be similar to cameras 130 and illumination units 710.

More precisely, the distal tip portion may include:
A circuit board assembly (CBA) including, on a distal section thereof, at least a front camera and two opposite-facing side-cameras configured to jointly provide a field-of-view (FOV) of at least about 270 degrees.
A heat sink structure, essentially as described above in the description of rigid shaft 102 (or as described in the description of shaft 1202 when the semi-rigid endoscope includes an active heat removal infrastructure).
One or more illumination units mounted on the heat sink structure and configured to illuminate the FOV.

The rigid member may be at least partially hollow with at least one semi-rigid conduit extending therethrough. The at least one semi-rigid conduit is thermally coupled to the heat sink structure, and thereby to the one or more illumination units. The at least one semi-rigid conduit extends proximally from the heat sink structure along the rigid member or a part of the rigid member. The at least one semi-rigid conduit is configured to be bent and/or twisted by the maneuvering portion, such as to allow the maneuvering of the distal tip portion.

According to some embodiments, wherein the semi-rigid endoscope includes a passive heat removal system, the at least one semi-rigid conduit may consist of a single semi-rigid conduit configured for heat conduction. According to some alternative embodiments, wherein the semi-rigid endoscope includes an active heat removal system, the at least one semi-rigid conduit may include pair of hollow semi-rigid conduit configured for circulating cooling fluid to, and from, the heat sink structure.

As used herein, according to some embodiments, the term “heat conduit” may refer to a conduit configured for heat conduction (e.g. made of a material characterized by high heat conductance). A metallic rod provides a (non-limiting) example of a heat conduit. According to some embodiments, in particular, embodiments wherein the endoscope does not incorporate an active heat removal system, the term “heat conduit” may be used interchangeably with “conduit”.

As used herein, according to some embodiments, the terms “first part” and “lower part” (e.g. sink lower part 402, sink lower part 1402) with respect to a heat sink structure, such as heat sink structure 400 or heat sink structure 1400, are interchangeable. Similarly, as used herein, according to some embodiments, the terms “second part” and “upper part” (e.g. sink upper part 404, sink upper part 1404) with respect to a heat sink structure, such as heat sink structure 400 or heat sink structure 1400, are interchangeable.

As used herein, according to some embodiments, the term “front” in reference to a component/element on or in an object may refer to a distally positioned (located) component/element on or in the object. Thus, for example, front camera 310a is positioned at, or in proximity to, the distal tip of shaft 102. Similarly, according to some embodiments, the term “back” in reference to a component/element on or in an object may refer to a proximally positioned component/element on or in the object. Thus, for example, back groove 526 is positioned in the proximal section of sink lower part 402.

As used herein, according to some embodiments, the terms “circuit board” and “printed circuit board” are interchangeable.

As used herein, according to some embodiments, the terms “first port” and “second port” with reference to ports on the handle (e.g. handle 1204) of an active heat removal endoscope (e.g. endoscope 1200) may be used interchangeably with “fluid-intake port” and “fluid-expulsion port”, respectively.

As used herein, the term “fluid” should be understood to encompass both liquid and gas.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

What it claimed is:

1.-37. (canceled)

38. An elongated shaft for a multi-camera rigid endoscope, the shaft comprising:
a circuit board assembly (CBA) comprising, on a distal section thereof, at least a front camera and two opposite-facing side-cameras configured to jointly provide a field-of-view (FOV) of at least about 270 degrees;
a heat sink structure located at a distal section of the shaft;
one or more illumination units mounted on the heat sink structure and configured to illuminate the FOV; and
at least one conduit configured for heat conveyance, the at least one conduit being thermally coupled to the heat sink structure, and thereby to the one or more illumination units, the at least one conduit extending proximally from the heat sink structure along the shaft or a part of the shaft,
wherein the heat sink structure is essentially cylindrical and comprises at least two complementary parts forming a compartment therebetween that accommodates the distal section of the CBA; and
wherein the heat sink structure comprises holes where through lens assemblies of the cameras are fitted, respectively, such as to secure a distal section of the CBA within the compartment.

39. The shaft of claim 38, wherein at least one of the holes is formed by corresponding grooves on the complementary parts.

40. The shaft of claim 38, wherein the complementary parts of the heat sink structure are metallic and welded onto one another, glued onto one another, and/or secured onto one another using screws.

41. The shaft of claim 38, wherein the two opposite-facing side-cameras comprise a first a side-camera and a second side-camera, and wherein the first side-camera and the second side-camera are positioned on the CBA such that a distance of the first side-camera to a distal end of the shaft is greater than a distance of the second side-camera to the distal end.

42. The shaft of claim 38, wherein the front camera is offset relative to a longitudinal axis of the shaft, which centrally extends along the shaft.

43. The shaft of claim 38, wherein the one or more illumination units comprise a front illumination unit and two side-illumination units, wherein the front illumination unit is mounted on a distal end of the heat sink structure, and wherein the heat sink structure comprises on opposite side-surfaces thereof two niches in which the two side illumination units are mounted, respectively.

44. The shaft of claim 43, wherein the front illumination unit is attached to the distal end of the heat sink structure, and wherein the two side illumination units are attached to the side-surfaces of the of the heat sink structure in the two niches, respectively.

45. The shaft of claim 44, wherein the front illumination unit is glued, screwed, and/or brazed with tin, to the distal end of the heat sink structure, and wherein the two side illumination units are glued, screwed, and/or brazed with tin to the side-surfaces of the of the heat sink structure in the two niches, respectively.

46. The shaft of claim 38, further comprising a rigid cover mounted on the heat sink structure and an elongated tube wherethrough the at least one conduit extends;
wherein the rigid cover comprises windows positioned over the lens assemblies and the illumination units; and
wherein the elongated tube is connected at a distal end thereof to a proximal end of the rigid cover, and wherein the connection is fluid tight such as to fluidly seal the distal section of the shaft.

47. The shaft of claim 46, wherein the rigid cover is made of stainless steel.

48. The shaft of claim 38, wherein the at least two complementary parts of the heat sink structure include a first part and a second part, each being shaped essentially as a half cylinder; and/or wherein the first part comprises an opening along the length thereof, and wherein the second part comprises a depression on an inner surface thereof, the depression and the opening forming the compartment of the heat sink structure.

49. The shaft of claim 48, wherein the CBA comprises a neck member connecting the distal section of the CBA to a proximal section of the CBA, and wherein the first part comprises, on an outer surface of a proximal section of the first part, a recess wherethrough the neck member extends.

50. The shaft of claim 38, wherein the at least one conduit consists of a single heat conduit, wherein the heat sink structure comprises a socket extending in the distal direction from a proximal end of the heat sink structure, and wherein a distal portion of the conduit is fitted into the socket, such as to thermally couple the heat conduit to the heat sink structure; and
wherein the heat conduit comprises a material characterized by high thermal conductance.

51. The shaft of claim 38, wherein the at least one conduit comprises two conduits, which are hollow and extend in parallel to one another, wherein the heat sink structure comprises a first channel and a second channel, each channel extending in the proximal direction from a proximal end of the heat sink structure, wherein first channel and the second channel are fluidly coupled to one another via respective distal ends thereof, and wherein the two conduits are fluidly connected to first channel and the second channel, respectively, the shaft being thereby configured for fluid conveyance through the distal section of the shaft.

52. The shaft of claim 51, wherein the heat sink structure further comprises a front channel positioned within the heat sink structure proximally to the distal end of the heat sink structure, the front channel fluidly couples the two channels; and
wherein the channels are included in the second part of the heat sink structure.

53. The shaft according to claim 52, wherein the first channel and the second channel, and, optionally, the front channel, are embedded in the heat sink structure.

54. An endoscope comprising a shaft according to claim 38 and a handle on which the shaft is mounted.

55. An endoscope comprising a shaft according to claim 51 and a handle on which the shaft is mounted.

56. The endoscope of claim 55, wherein the shaft comprises the two conduits and the first channel and the second channel, wherein the two conduits comprise a first conduit and a second conduit, wherein the handle comprises a fluid reservoir and a pump, wherein the fluid reservoir is fluidly coupled to the two conduits, and wherein the pump is configured to circulate fluid from the fluid reservoir by pumping fluid into the first conduit and drawing fluid from the second conduit.

57. The endoscope of claim 55, wherein the shaft comprises the two conduits and the first channel and the second channel, wherein the two conduits comprise a first conduit and a second conduit, wherein the handle further comprises a fluid-intake port and a fluid-expulsion port, and wherein the first conduit is fluidly coupled to the fluid-intake port and the second conduit is fluidly coupled to the fluid-expulsion port.\
